(19) United States
(12) Patent Application Publication
(10) Pub. No.: US 2022/0227726 A1
(43) Pub. Date: Jul. 21, 2022

(54) CANNABINOID GLYCOSIDES AND USES THEREOF

(71) Applicant: Graphium Biosciences, Inc., Cleveland, OH (US)

(72) Inventors: Janee' M. HARDMAN, Fair Oaks, CA (US); Brandon J. ZIPP, Roseville, CA (US); Tyrel R. DEUTSCHER, Wauwatosa, WI (US)

(73) Assignee: Graphium Biosciences, Inc., Cleveland, OH (US)

(21) Appl. No.: 17/510,817

(22) Filed: Oct. 26, 2021

Related U.S. Application Data
(63) Continuation of application No. PCT/US2020/019886, filed on Feb. 26, 2020.

(51) Int. Cl. C07D 311/80; C07D 407/12; C07D 407/14

(52) U.S. Cl. CPC C07D 311/80; C07D 407/14; C07D 407/12

(57) ABSTRACT

The present invention provides tetrahydrocannabinol glycoside and cannabidiol glycoside prodrugs and pharmaceutical compositions comprising these compounds, and their use for the site specific delivery of tetrahydrocannabinol or cannabidiol. Also provided are processes for the preparation of purified tetrahydrocannabinol glycoside and cannabidiol glycoside prodrugs.

### FIELD OF THE INVENTION

The present invention pertains to the field of drug development and in particular to novel cannabinoid glycoside prodrugs.

### BACKGROUND

Phytocannabinoids from Cannabis sativa have long been used for altering mental states, but recent findings have illuminated the potential of specific cannabinoid compounds for treatment and maintenance of various diseases and conditions.

Cannabinoids are extremely hydrophobic in nature, complicating their use in drug formulations. Non-covalent methods have been found to improve the solubility of cannabinoids by utilizing carrier carbohydrates such as cyclized maltodextrins (Jarho 1998). Covalent chemical manipulations have produced novel CBD prodrugs with improved solubility (WO 2009/018389, WO 2012/011112). Even fluorine substituted CBD compounds have been created through synthetic chemical manipulations in an effort to functionalize CBD (WO 2014/108899). The aforementioned strategies were somewhat successful in improving the solubility of CBD, but they create unnatural compositions which alter the composition and will release the unnatural prodrug moieties upon hydrolysis.

Of particular interest is the psychotropic molecule tetrahydrocannabinol (THC). THC is an agonist of the cannabinoid 1 (CB1) receptors in the brain and binding produces a high or sense of euphoria. THC has potential application in treating conditions such as pain, glaucoma, insomnia, low appetite, nausea, anxiety and muscle spasticity.

One shortcoming of THC is that its extremely hydrophobic nature makes it difficult for formulation and delivery. Additionally, current pharmaceutical compositions of THC have unpleasant organoleptic properties, and their hydrophobic nature results in a lingering on the palate.

A growing body of evidence shows that glycosides are capable of acting as prodrugs and also may have direct therapeutic effects. Site-specific delivery of steroid glycosides to the colon has previously been demonstrated (Friend 1985, Friend 1984), and could enable treatment of local disorders such as inflammatory bowel disease. Glycosylation of steroids enabled survival of stable bioactive molecules in the acidic stomach environment and delivery into the large intestine, where the aglycones were liberated by glycosidases produced by colonic bacteria, and then absorbed into the systemic circulation. Glycosidases are also present universally in different tissues (Conchie 1959), so delivery of glycosides by methods that bypass the digestive tract and colon, such as intravenous delivery, will enable targeted delivery to other cells and tissues that have increased expression of glycosidases. In addition, the distribution of alpha-glycosidase and beta-glycosidase enzymes differ throughout the intestinal tract and other tissues, and different forms of glycosides may therefore provide unique pharmacokinetic profiles, including formulations that target delivery of specific diseased areas, or targeted release at locations that can promote or restrict systemic absorption of the cannabinoids and other compounds described herein. Many biologically active compounds are glycosides, including members of classes of compounds such as hormones, antibiotics, sweeteners, alkaloids, and flavonoids. While it is generally accepted that glycosides will be more water-soluble than the aglycones, literature reviews have analyzed structure-activity relationships and determined that it is nearly impossible to define a general pattern for the biological activities of glycosides across different classes of compounds (Kren 2008).

Cannabinoid glycosides available as cannabinoid prodrugs are known from PCT application WO 2017/053574, which discloses a method for the efficient regioselective production of cannabinoid glycosides using glucosyltransferase enzymes, which allows for the production of large quantities of individual glycosides. This reference also disclosed the assessment of selected cannabinoid glycosides for their pharmaceutical properties, including evaluation of in vivo drug pharmacokinetics and pharmacodynamics to identify cannabinoid glycosides as potential prodrugs of cannabinoids, and as novel cannabinoid compositions with novel properties and functions.

Although select cannabinoid glycosides have been shown to have different pharmacokinetic and pharmacodynamic properties than the bare cannabinoid molecules, there is a need for novel cannabinoid prodrugs that can be tailored to provide specific drug bioavailability or pharmacokinetic properties, including improved site-specific or tissue-specific drug delivery, better than previously known cannabinoid glycosides.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide novel cannabinoid glycosides and uses thereof. In accordance with an aspect of the present invention, there is provided a tetrahydrocannabinol glycoside prodrug compound having Formula (I):

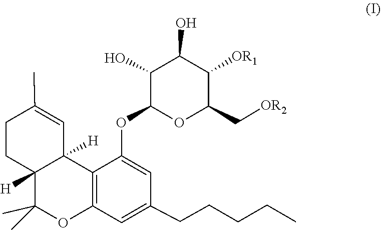

wherein R₁ is H, β-D-glucopyranosyl, or 3-O-β-D-glucopyranosyl-β-D-glucopyranosyl; and R₂ is H or β-D-glucopyranosyl; with the proviso that R₁ and R₂ are not both H.

In accordance with another aspect of the present invention, there is provided a cannabidiol glycoside prodrug compound having Formula (II):

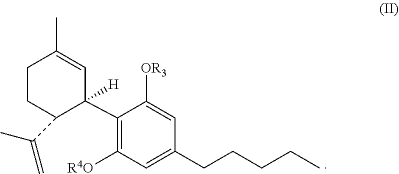

wherein R₃ and R₄ are H or a moiety having the structure:

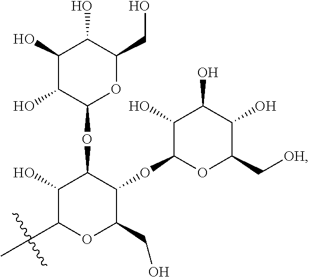 [SMILES: *C1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]

with the proviso that R₃ and R₄ are not both H.

In accordance with another aspect of the present invention, there is provided a pharmaceutical composition comprising a tetrahydrocannabinol glycoside prodrug of the present invention, and a pharmaceutically acceptable carrier, diluent, excipient, or adjuvant.

In accordance with another aspect of the present invention, there is provided a method for the site-specific delivery of tetrahydrocannabinol to the intestinal lumen of a subject, comprising the step of administering a tetrahydrocannabinol glycoside prodrug to a subject in need thereof.

In accordance with another aspect of the present invention, there is provided a method for the site-specific delivery of tetrahydrocannabinol to the intestinal lumen of a subject, comprising the step of administering a pharmaceutical composition comprising a tetrahydrocannabinol glycoside prodrug to a subject in need thereof.

In accordance with another aspect of the present invention, there is provided a pharmaceutical composition comprising a cannabidiol glycoside prodrug of the present invention, and a pharmaceutically acceptable carrier, diluent, excipient, or adjuvant.

In accordance with another aspect of the present invention, there is provided a method for the site-specific delivery of cannabidiol to the intestinal lumen of a subject, comprising the step of administering a cannabidiol glycoside prodrug to a subject in need thereof.

In accordance with another aspect of the present invention, there is provided a method for the site-specific delivery of cannabidiol to the intestinal lumen of a subject, comprising the step of administering a pharmaceutical composition comprising a cannabidiol glycoside prodrug to a subject in need thereof

In accordance with another aspect of the present invention, there is provided a process for the preparation of a purified cannabinoid glycoside prodrug comprising the steps of: (a) providing a mixture of higher order cannabinoid glycosides; (b) incubating the mixture of cannabinoid glycosides with at least one hydrolase enzyme for a period of time sufficient to hydrolyze at least a portion of the glycosidic bonds to form a refined mixture of cannabinoid glycosides; and (c) separating the purified cannabinoid glycoside prodrug from the refined mixture of cannabinoid glycosides.

### BRIEF DESCRIPTION OF THE FIGURES

FIG. 1(a) is a graphical representation of binding data for VB302 and Δ9-THC in the human cannabinoid receptor type 1 (CB1R).

FIG. 1(b) is a graphical representation of binding data for VB302 and Δ9-THC in the human cannabinoid receptor type 2 (CB2R).

FIG. 2 is a graphical representation of cannabinoid plasma concentrations following oral administration of VB302.

FIG. 3(a) is a tabular summary of digestion activity observed in screening assays for a panel of commercially available glycoside hydrolases.

FIG. 3(b) is a tabular summary of the resulting THC-glycoside products observed in screening assays for a panel of commercially available glycoside hydrolases.

FIG. 4(a) is a graphical representation of the starting THC-glycoside mixture VB300X before undergoing in vitro enzymatic digestion.

FIG. 4(b) is a graphical representation of the digestion product of VB300X after digestion with Vinotase Pro enzyme.

FIG. 4(c) is a graphical representation of the digestion product of VB300X after digestion with Lallzyme Beta™ enzyme.

FIG. 5(a) is a graphical representation of a CBD-glycoside mixture of VB112 and VB119 before undergoing in vitro enzymatic digestion.

FIG. 5(b) is a graphical representation of the digestion product of a mixture of VB112 and VB119 after digestion with Vinotase Pro™ enzyme.

FIG. 5(c) is a graphical representation of the digestion product of a mixture of VB112 and VB119 after digestion with Lallzyme Beta™ enzyme.

FIG. 6(a) Is a tabular summary of the results of the digestion assays using biological samples as hydrolase sources.

FIG. 6(b) is a tabular summary of the products of the digestion assays using biological samples as hydrolase sources.

FIGS. 7(a)-(d) are graphical representations of the relative amounts of THC-glycosides and metabolites present in the plasma of rats at 1, 2, 6 and 24 hour post administration of VB311 by oral gavage, respectively.

FIG. 8 illustrates possible decoupling pathways of THC-glycosides.

FIGS. 9(a) and (b) illustrate possible decoupling pathways of CBD-glycosides.

FIG. 10(a) is a graphical representation of plasma Cmax values of VB302 and VB311 in rats post administration by oral gavage.

FIG. 10(b) is a graphical representation of average area under the curve (AUC) values for VB302 and VB311 in plasma in rats post administration by oral gavage.

FIG. 10(c) is a graphical representation of plasma Cmax values of THC in rats post administration VB302 or VB311 by oral gavage.

FIG. 10(d) is a graphical representation of average area under the curve (AUC) values for THC in plasma in rats post administration of VB302 or VB311 by oral gavage.

FIG. 11(a) is a graphical representation of the distribution of THC-glycosides in a glycoside mixture administered to rats by oral gavage, shown as normalized average peak area undor the ourvc (AUC) of total glycosides.

FIGS. 11(b) and 11(c) are graphical representations of THC-glycosides and metabolites present in biological samples obtained from rats post administration of THC-glycoside by oral gavage, shown as normalized average peak area under the curve (AUC) of total glycosides.

FIG. 12(a) is a graphical depiction of the relative amounts of a final mixture of CBD-glycosides following incubation of VB135 with Lallzyme Beta™ (Lallemand).

FIG. 12(b) is a graphical depiction of the relative amounts of a final mixture of CBD-glycosides following incubation of VB135 with Vinotaste Pro (Novozymes).

FIGS. 13(a) and 13(b) are graphical depictions of the change in the amount of CBD glycosides present over the course of a fecal digestion study.

### DETAILED DESCRIPTION OF THE INVENTION

The abbreviations “Δ9-THC” and “THC” are used interchangeably and refer to Δ-9 tetrahydrocannabinol or tetrahydrocannabinol.

The term “glucopyranoside” is used for naming molecules and is shorthand for a β-D-glucose attached through the hydroxyl at the 1-position (the anomeric carbon) of the glucose to an aglycone or another glucose residue.

The term “aglycone” is used in the present application to refer to the non-glycosidic portion of a glycoside compound.

The term “prodrug” refers to a compound that, upon administration, must undergo a chemical conversion by metabolic processes before becoming an active pharmacological agent.

The term “cannabinoid glycoside prodrug” refers to glycosides of a cannabinoid aglycone. A glycoside prodrug undergoes hydrolysis of the glycosidic bond, typically by action of a glycosidase, to release the active cannabinoid aglycone to a desired site in the body of the subject.

The term “tetrahydrocannabinol glycoside prodrug” refers to glycosides of the cannabinoid tetrahydrocannabinol (THC).

The term “cannabidiol glycoside prodrug” refers to glycosides of the cannabinoid cannabidiol (CBD).

The expression “higher glycosides” or “higher order glycosides” refers to glycosides having two or more sugar residues. A higher glycoside may have the two or more sugar residues in a branched or linear configuration.

The term “recalcitrance” refers to the resistance of a chemical structure or carbohydrate configuration to break down or be metabolized.

The term “subject” or “patient” as used herein refers to an animal in need of treatment. In one embodiment, the animal is a human.

As used herein, the term “about” refers to a +/−10% variation from the nominal value. It is to be understood that such a variation is always included in a given value provided herein, whether or not it is specifically referred to.

Unless defined otherwise, all technical and scientific terms used herein have the same meaning as commonly understood by one of ordinary skill in the art to which this invention belongs.

In accordance with the present invention, the THC-glycoside and CBD-glycoside prodrugs are converted upon hydrolysis of the glycosidic bond to provide the active cannabinoid drug. Accordingly, the present invention has demonstrated that glycosides with a hydrophobic aglycone moiety undergo glucose hydrolysis in the gastrointestinal tract or in tissues having increased expression of glycosidases, yielding the hydrophobic tetrahydrocannabinol or cannabidiol compound in the targeted tissue or organ.

The glucose residues of glycosides can be cleaved by glycosidase enzymes in the intestinal tract, including by alpha-glycosidases and beta-glycosidases, which are expressed by intestinal microflora across different regions of the intestine. Accordingly, glycosides are hydrolyzed upon ingestion to release the desired compound into the intestines or target tissues.

In one embodiment, glycosylation of tetrahydrocannabinol (THC) provides tetrahydrocannabinol glycoside prodrugs (THC-glycoside prodrugs) capable of persisting in the acidic stomach environment upon oral administration, thereby allowing delivery of the prodrug into the large intestine, where the THC aglycone can be liberated by glycosidases produced by colonic bacteria.

In one embodiment, the THC-glycoside prodrugs are suitable for targeted delivery to tissues having increased expression of glycosidases. Upon parenteral administration of the THC-glycoside prodrug formulation to the subject, the THC aglycone is liberated by the glycosidases in the target tissues.

In one embodiment, glycosylation of cannabidiol (CBD) provides cannabidiol glycoside prodrugs (CBD-glycoside prodrugs) capable of persisting in the acidic stomach environment upon oral administration, thereby allowing delivery of the prodrug into the large intestine, where the CBD aglycone can be liberated by glycosidases produced by colonic bacteria.

In one embodiment, the CBD-glycoside prodrugs are suitable for targeted delivery to tissues having increased expression of glycosidascs. Upon parenteral administration of the CBD-glycoside prodrug formulation to the subject, the CBD aglycone is liberated by the glycosidases in the target tissues.

In one embodiment, the THC-glycoside and/or CBD-glycoside prodrugs can be administered with a substance that has direct glycosidase activity or that may in other ways alter the prodrug metabolism and pharmacokinetic profile. Upon interaction of the prodrug and substance with glycosidase activity, the THC and/or CBD aglycone is liberated by the glycosidases in the target tissue.

In one embodiment, the tetrahydrocannabinol base molecule of the cannabinoid-glycoside may be Δ8-tetrahydrocannabinol (Δ8-THC). In one embodiment, the cannabinoid base molecule of the cannabinoid-glycoside may be tetrahydrocannabidavarin (THCV). In one embodiment, the cannabinoid base molecule of the cannabinoid-glycoside is the carboxylated form of THC, tetrahydrocannabinol acid (THCA). In other embodiments, the cannabinoid base molecule of the cannabinoid-glycoside may be cannabidivarin (CBDV). In other embodiments, the cannabinoid base molecule of the cannabinoid-glycoside may be cannabinol (CBN). In other embodiments, the cannabinoid base molecule of the cannabinoid-glycoside may be cannabigerol (CBG). In one embodiment, the cannabinoid base molecule of the cannabinoid-glycoside may be an endocannabinoid.

It is also within the scope of the present invention that the THC-glycoside and CBD-glycoside prodrugs are also useful as pharmaceutical agents, where they exhibit novel pharmacodynamic properties compared to the parent compound alone. The increased aqueous solubility of the THC-glycoside and CBD-glycoside prodrugs of the present invention also enables new formulations for delivery in transdermal or aqueous formulations that would not have been achievable if formulating hydrophobic cannabinoid molecules.

The present invention relates to novel tetrahydrocannabinol-based and cannabidiol-based glycoside prodrugs and methods for their use for the site-specific delivery of tetrahydrocannabinol or cannabidiol to a subject.

In one embodiment of the present invention, there are provided tetrahydrocannabinol glycoside prodrug compounds having Formula (I):

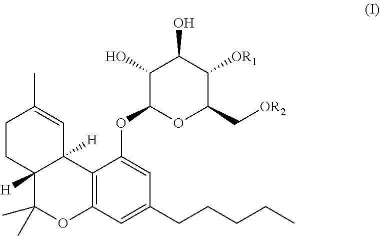

wherein R₁ is H, β-D-glucopyranosyl, or β-O-β-D-glucopyranosyl-β-D-glucopyranosyl; and R₂ is H or β-D-glucopyranosyl, with the proviso that R₁ and R₂ are not both H.

Exemplary tetrahydrocannabinol (THC)-glycosides falling within the scope of Formula (I), include:

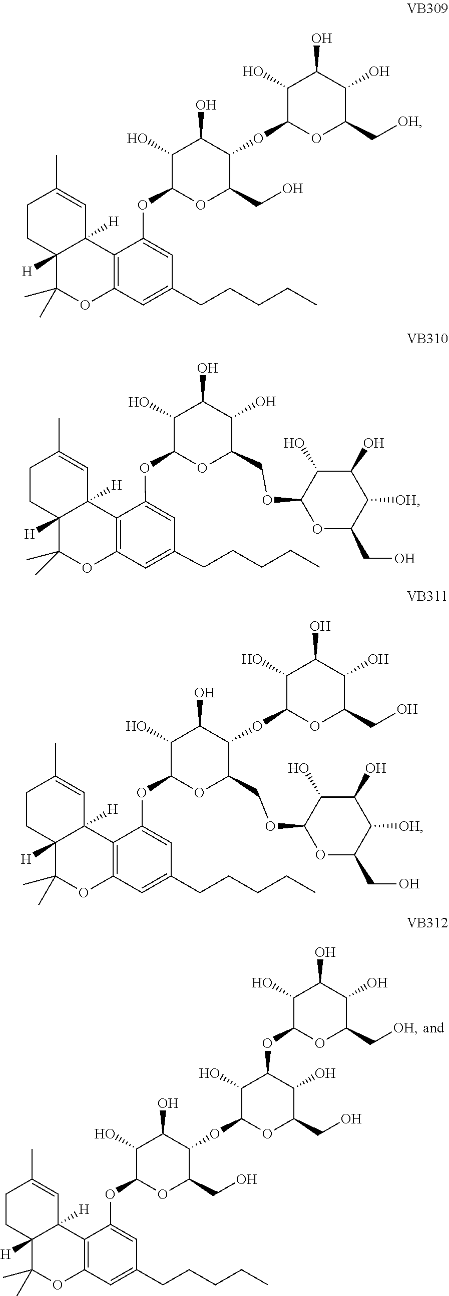

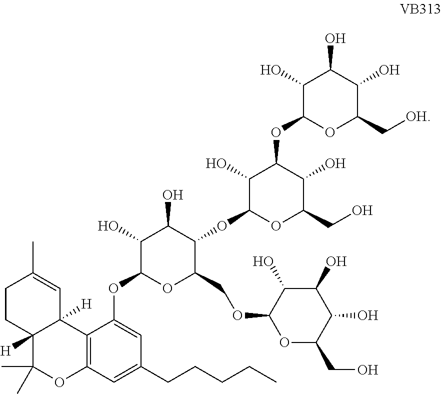 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O[C@@H]4O[C@H](CO)[C@@H](O)[C@H](O)[C@H]4O)[C@H]3O)[C@H](O)[C@H]2O)c2c(c1)OC(C)(C)[C@@H]1CCC(C)=C[C@@H]21]

In a preferred embodiment, the tetrahydrocannabinol glycoside prodrug is

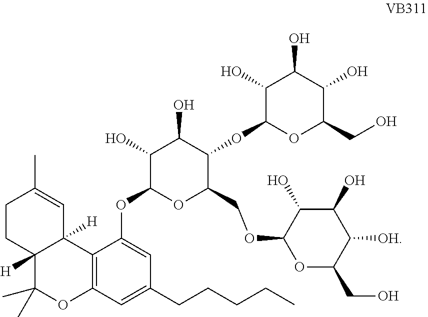 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]

In one embodiment of the present invention, there are provided cannabidiol glycoside prodrug compounds having Formula (II):

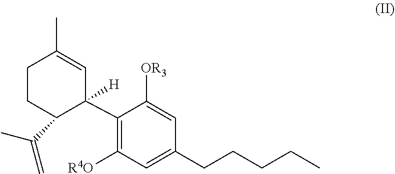

wherein R₃ and R₄ are H or a moiety having the structure:

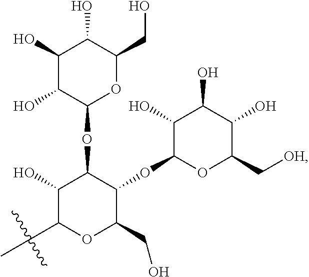 [SMILES: *C1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]

with the proviso that R₃ and R₄ are not both H.

Exemplary cannabidiol (CBD)-glycosides falling within the scope of Formula (II), include

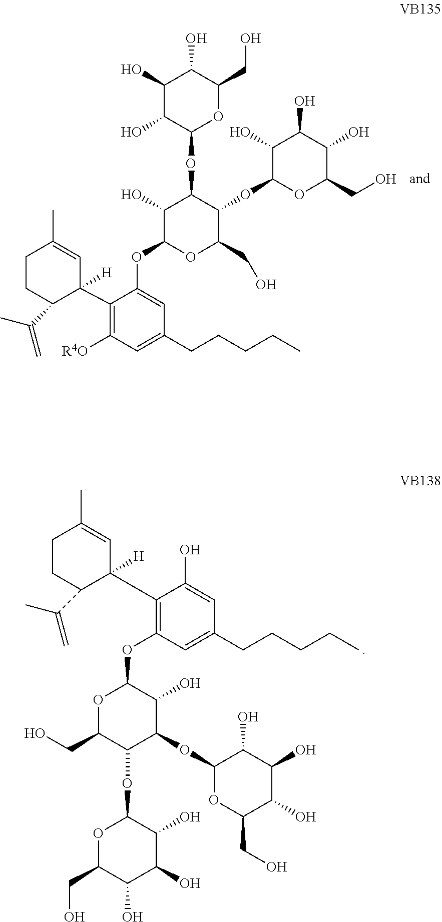

In one embodiment, there is provided a method for the site-specific delivery of a THC or CBD drug to a subject, comprising the step of administering to a subject in need thereof one or more THC-glycoside or CBD-glycoside prodrugs in accordance with the present invention. In one embodiment, the site of delivery is the large intestine. In one embodiment, the site of delivery is the rectum. In one embodiment, the site of delivery is the liver. In one embodiment, the site of delivery is the skin. In one embodiment, the site of delivery is the eye.

In one embodiment, there is provided a method for facilitating the transport of THC or CBD to the brain through intranasal, stereotactic, or intrathecal delivery, or delivery across the blood brain barrier of a subject comprising administering a THC-glycoside or CBD-glycoside prodrug in accordance with the present invention to a subject in need thereof.

In one embodiment, there is provided a method for the site-specific delivery of a cannabidiol drug to a subject, comprising the step of administering to a subject in need thereof one or more CBD-glycoside prodrugs in accordance with the present invention. In one embodiment, the site of delivery is the large intestine. In one embodiment, the site of delivery is the rectum. In one embodiment, the site of delivery is the liver. In one embodiment, the site of delivery is the skin. In one embodiment, the site of delivery is the eye.

In one embodiment, there is provided a method for delayed or extended release of the cannabinoid aglycone for sustained delivery of the compound for therapeutic use.

In accordance with the present invention, the THC-glycoside or CBD-glycoside prodrugs are useful in the treatment of conditions that benefit from and/or can be ameliorated with the site-specific administration of THC or CBD. Conditions that can be treated and/or ameliorated through the administration of THC-glycoside or CBD-glycoside prodrugs of the present invention, include but are not limited to, inflammatory bowel disease including induction of remission from Crohn's disease, and colitis and induction of remission from ulcerative colitis. Among the benefits that can be achieved through the administration of THC-glycoside and/or CBD-glycoside prodrugs of the present invention are decreased inflammation of the intestines and rectum, decreased pain in the intestines, rectum, as well as decrease in neuropathic pain and abdominal pain, antimicrobial action in the intestines, and inhibition of proliferation or cytotoxicity against colorectal cancer. Additional treatment indications, effects, or applications for THC-glycosides or CBD-glycosides may include but are not limited to anorexia, nausea, emesis, pain, wasting syndrome, HIV-wasting, chemotherapy induced nausea and vomiting, epilepsy, schizophrenia, irritable bowel syndrome, cramping, spasticity, seizure disorders, alcohol use disorders, substance abuse disorders, addiction, cancer, amyotrophic lateral sclerosis, glioblastoma multiforme, glioma, increased intraocular pressure, glaucoma, cannabis use disorders, Tourette's syndrome, dystonia, multiple sclerosis, white matter disorders, demyelinating disorders, chronic traumatic encephalopathy, leukoencephalopathies, Guillain-Barre syndrome, inflammatory bowel disorders, gastrointestinal disorders, bacterial infections, Methicillin-resistant Staphylococcus aureus (MRSA), Clostridioides difficile (formerly Clostridium difficile, or C. diff.), sepsis, septic shock, viral infections, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, neuropathic pain, neuropathic pain associated with post-herpetic neuralgia, diabetic neuropathy, shingles, burns, actinic keratosis, oral cavity sores and ulcers, post-episiotomy pain, psoriasis, pruritis, gout, chondrocalcinosis, joint pain, fibromyalgia, musculoskeletal pain, neuropathic-postoperative complications.

In one embodiment, the THC-glycoside prodrugs can be used in the treatment and/or amelioration of inflammatory bowel disease. In another embodiment, the THC-glycoside prodrugs can be used in the treatment and/or amelioration of Crohn's disease. In another embodiment, the THC-glycoside prodrugs can be used in the treatment and/or amelioration of colitis. In some embodiments, the colitis is ulcerative colitis. In another embodiment, the THC-glycoside prodrugs can be used for the induction of remission from ulcerative colitis.

In one embodiment, the cannabinoid-glycoside prodrug is administered in a pharmaceutical composition further comprising a pharmaceutically acceptable carrier, diluent, excipient, or adjuvant. In one embodiment, the pharmaceutical compositions comprise one or more cannabinoid-glycoside prodrugs and one or more pharmaceutically acceptable carriers, diluents, excipients and/or adjuvants. For administration to a subject, the pharmaceutical compositions can be formulated for administration by a variety of routes including but not limited to oral, topical, rectal, parenteral, and intranasal administration.

The pharmaceutical compositions may comprise from about 1% to about 95% of a cannabinoid-glycoside prodrug of the invention. Compositions formulated for administration in a single dose form may comprise, for example, about 20% to about 90% of the cannabinoid-glycoside prodrug of the invention, whereas compositions that are not in a single dose form may comprise, for example, from about 5% to about 20% of the cannabinoid-glycoside prodrug of the invention. Non-limiting examples of unit dose forms include tablets, ampoules, dragees, suppositories, and capsules.

In a preferred embodiment, the pharmaceutical compositions are formulated for oral administration. Pharmaceutical compositions for oral administration can be formulated, for example, as tablets, troches, lozenges, aqueous or oily suspensions, dispersible powders or granules, emulsion hard or soft capsules, or syrups or elixirs. Such compositions can be prepared according to standard methods known in the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in an admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed to further facilitate delivery of the drug compound to the desired location in the digestive tract.

Pharmaceutical compositions for oral use can also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Pharmaceutical compositions formulated as aqueous suspensions contain the active compound(s) in an admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose, stevia, or saccharin.

Pharmaceutical compositions can be formulated as oily suspensions by suspending the active compound(s) in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

The pharmaceutical compositions can be formulated as a dispersible powder or granules, which can subsequently be used to prepare an aqueous suspension by the addition of water. Such dispersible powders or granules provide the active ingredient in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Pharmaceutical compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Pharmaceutical compositions can be formulated as a syrup or elixir by combining the active ingredient(s) with one or more sweetening agents, for example glycerol, propylene glycol, sorbitol or sucrose. Such formulations can also optionally contain one or more demulcents, preservatives, flavouring agents and/or colouring agents.

If desired, other active ingredients may be included in the compositions. In one embodiment, the glycoside prodrugs may be combined with other ingredients or substances that have glycosidase activity, or that may in other ways alter drug metabolism and pharmacokinetic profile of various compounds in vivo, including ones in purified form as well as such compounds found within food, beverages, and other products. In one embodiment, the THC-glycoside prodrug is administered in combination with, or formulated together with, substances that have direct glycosidase activity, or that contribute to modifications to the gut microflora that will alter the glycosidase activity in one or more regions of the intestines. Examples of such compositions include, but are not limited to, yogurt, prebiotics, probiotics, or fecal transplants.

In some embodiments, the glycosidase ingredient or substance that has glycosidase activity may be administered directly with the THC-glycoside and/or CBD-glycoside prodrug. In other embodiments, the glycosidase ingredient or substance that has glycosidase activity may be administered separately from the THC-glycoside and/or CBD-glycoside prodrug. In one embodiment, the glycosidase ingredient or substance that has glycosidase activity may be administered before the THC-glycoside and/or CBD-glycoside prodrug. In one embodiment, the glycosidase ingredient or substance that has glycosidase activity may be administered after the THC-glycoside and/or CBD-glycoside prodrug. In one embodiment, the glycosidase ingredient or substance that has glycosidase activity made be formulated such that it is released in a time or environmental dependent manner (for example, delayed release, sustained release, release dependant on pH or other environmental factor).

In one embodiment, the glycosidase ingredient or substance is an enzyme having glycolytic activity. In some embodiments, the glycosidase ingredient or substance is a broadly active beta-glucosidase. In some embodiments, the glycosidase ingredient or substance is a beta-glucosidase from almonds, Lallzyme Beta™, Vinotaste Pro, or combinations thereof.

In a further preferred embodiment, the pharmaceutical compositions are formulated for parenteral administration. The term “parenteral” as used herein includes subcutaneous injections, intravenous, intramuscular, intrathecal, intrasternal injection or infusion techniques.

Parenteral pharmaceutical compositions can be formulated as a sterile injectable aqueous or oleaginous suspension according to methods known in the art and using one or more suitable dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Due to the highly lipophilic nature of cannabinoids such as THC, these molecules are typically poorly absorbed through membranes such as the skin of mammals, including humans, and the success of transdermally administering therapeutically effective quantities of THC to a subject in need thereof within a reasonable time frame and over a suitable surface area has been substantially limited. It is therefore proposed that the THC-glycoside prodrugs of the present invention, through conjugation of the hydrophobic THC aglycone to the hydrophilic glycosidic moieties, provide a molecule having an amphiphilic character favourable for passive diffusion which should be more readily absorbed through the skin.

Accordingly, in one embodiment, the pharmaceutical compositions are formulated for topical administration. Such topical formulations may be presented as, for example, aerosol sprays, powders, sticks, granules, creams, liquid creams, pastes, gels, lotions, ointments, on sponges or cotton applicators, or as a solution or a suspension in an aqueous liquid, a non-aqueous liquid, an oil-in-water emulsion, or a water-in-oil liquid emulsion.

Topical pharmaceutical compositions can be formulated with thickening (gelling) agents. The thickening agent used herein may include anionic polymers such as polyacrylic acid(CARBOPOL® by Noveon, Inc., Cleveland, Ohio), carboxypolymethylene, carboxymethylcellulose and the like, including derivatives of Carbopol® polymers, such as Carbopol® Ultrez 10, Carbopol® 940, Carbopol® 941, Carbopol® 954, Carbopol® 980, Carbopol® 981, Carbopol® ETD 2001, Carbopol® EZ-2 and Carbopol® EZ-3, and other polymers such as Pemulen® polymeric emulsifiers, and Noveon® polycarbophils. Thickening agents or gelling agents are present in an amount sufficient to provide the desired rheological properties of the composition.

Topical pharmaceutical compositions can be formulated with a penetration enhancer. Non-limiting examples of penetration enhancing agents include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C22 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxyl)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The topical pharmaceutical compositions can further comprise wetting agents (surfactants), lubricants, emollients, antimicrobial preservatives, and emulsifying agents as are known in the art of pharmaceutical formations.

Transdermal delivery of the THC-glycoside prodrug can be further facilitated through the use of a microneedle array drug delivery system.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

The pharmaceutical compositions of the present invention described above include one or more THC-glycoside prodrugs of the invention in an amount effective to achieve the intended purpose. Thus the term “therapeutically effective dose” refers to the amount of the THC-glycoside prodrug that improves the status of the subject to be treated, for example, by ameliorating the symptoms of the disease or disorder to be treated, preventing the disease or disorder, or altering the pathology of the disease. Determination of a therapeutically effective dose of a compound is well within the capability of those skilled in the art. In one embodiment, THC-glycosides can be combined to enable simultaneous delivery with other cannabinoids in a site-specific manner, for example, CBD, whose effects in some ways may be synergistic (Russo 2006). Accordingly, in one embodiment, the pharmaceutical composition comprises one or more THC-glycosides and one or more CBD-glycosides formulated together in a single dosage form.

The exact dosage to be administered to a subject can be determined by the practitioner, in light of factors related to the subject requiring treatment. Dosage and administration are adjusted to provide desired levels of the THC-glycoside prodrug and/or the THC compound itself obtained upon hydrolysis of the prodrug. Factors which may be taken into account when determining an appropriate dosage include the severity of the disease state, general health of the subject, age, weight, and gender of the subject, diet, microbiota diversity and quantity, time and frequency of administration, route of administration, drug combination(s), reaction sensitivities, and tolerance/response to therapy. Dosing regimens can be designed by the practitioner depending on the above factors as well as factors such as the half-life and clearance rate of the particular formulation.

In some embodiments, the dosage of THC-glycoside prodrug is 0.0001 mg/kg to 10 mg/kg. In further embodiments, the dosage of the THC-prodrug is 0.0001 mg/kg to 1 mg/kg. In further embodiments, the dosage of the THC-prodrug is 0.0001 mg/kg to 0.1 mg/kg. In further embodiments, the dosage of the THC-prodrug is 0.0001 mg/kg to 0.01 mg/kg. In another embodiment, the dosage of the THC-prodrug is 0.0025 mg/kg.

In some embodiments, the dosage of THC-glycoside prodrug is equivalent to 0.00004 mg/kg to 4 mg/kg of THC. In further embodiments, the dosage of the THC-prodrug is equivalent to 0.00004 mg/kg to 0.4 mg/kg of THC. In further embodiments, the dosage of the THC-prodrug is equivalent to 0.00004 mg/kg to 0.04 mg/kg of THC. In further embodiments, the dosage of the THC-prodrug is equivalent to 0.00004 mg/kg to 0.004 mg/kg of THC. In further embodiments, the dosage of the THC-prodrug is equivalent to 0.00004 mg/kg to 0.0004 mg/kg of THC. In another embodiment, the dosage of THC-prodrug is equivalent to 0.001 mg/kg THC.

In some embodiments, the dosage of CBD-glycoside prodrug is 0.001 mg/kg to 100 mg/kg. In further embodiments, the dosage of the CBD-prodrug is 0.001 mg/kg to 10 mg/kg. In further embodiments, the dosage of the CBD-prodrug is 0.001 mg/kg to 1 mg/kg. In further embodiments, the dosage of the CBD-prodrug is 0.001 mg/kg to 0.1 mg/kg. In another embodiment, the dosage of the CBD-prodrug is 0.025 mg/kg.

In some embodiments, the dosage of CBD-glycoside prodrug is equivalent to 0.0004 mg/kg to 4 mg/kg of CBD. In further embodiments, the dosage of the CBD-prodrug is equivalent to 0.0004 mg/kg to 0.4 mg/kg of CBD. In further embodiments, the dosage of the CBD-prodrug is equivalent to 0.0004 mg/kg to 0.04 mg/kg of CBD. In another embodiment, the dosage of CBD-prodrug is equivalent to 0.01 mg/kg CBD.

In some embodiments, the THC-glycoside or CBD-glycoside prodrugs maybe be administered between once and three times a day. In some embodiments, the THC-glycoside or CBD-glycoside prodrugs may be administered once a day. In another embodiment the THC-glycoside or CBD-glycoside prodrugs may be administered twice a day.

The invention will now be described with reference to specific examples. It will be understood that the following examples are intended to describe embodiments of the invention and are not intended to limit the invention in any way.

### Structural Characterization of Compounds

The masses of the THC-glycosides were determined by LC-MS. LC separation was performed on a 3 μm ACE C18-PFP column using mobile phases of 0.1% formic acid in H₂O and acetonitrile w/ 0.1% formic acid.

Mass characterization was carried out by ESI mass spectrometry on an API4000 QTrap in both positive and negative modes. Infusion of compounds in 50:50 MeOH:H₂O shows preferential Na adduct formation. Sodium ions come from labware and were therefore uncontrolled so 5 mM ammonium formate was added to displace the Na adducts (M+23)⁺ with NH₄ adducts (M+18)⁺.

Structural characterization of each THC-glycosides was determined by 1D and 2D NMR analysis, including ¹H, ¹³C, DEPT-135, COSY, H2BC, HMBC, and HSQC. Spectra were recorded on a Varian Inova 500 in DMSO-d6, using Vnmrj 4.2a acquisition software and NUTS and/or SpinWorks 4.0 processing software.

Structural data for the THC aglycone VB301 and the THC-monoside VB302 are included to support interpretation of the NMR datasets for the higher-glycosides presented herein.

### Reference Data for VB301 (THC Aglycone)

The synthetic THC used as input for glycosylation was commercially purchased. THC was characterized by LC-MS and ¹H and ¹³C NMR to verify mass and determine chemical shift values of the aglycone.

LC-MS. [M+H]⁺ (C₂₁H₂₁O₂) Calcd: m/z=315.2. Found: m/z=315.5. [M+H]⁺ (C₂₁H₂₉O₂) Calcd: m/z=313.2. Found: m/z=313.5:

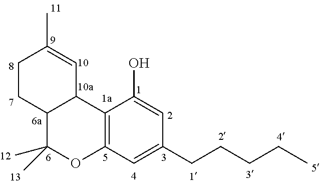 [SMILES: CCCCCc1cc(O)c2c(c1)OC(C)(C)C1CCC(C)=CC21]

TABLE 1
¹H (600 MHz) and ¹³C (150 MHz) NMR spectroscopic data
of VB301 (THC aglycone) (solvent: DMSO-d6)
VB301 (THC)

| Position | δ_{C} | Δ_{H} (J in Hz) |
|---|---|---|
| 1 | 156.49 | |
| 1a | 108.94 | |
| 1-OH | | 9.20 (s, 1H) |
| 2 | 107.61 | 6.36 (s, 1H) |
| 3 | 141.81 | |
| 4 | 108.33 | 6.00 (s, 1H) |
| 5 | 154.61 | |
| 6 | 76.84 | |
| 6a | 46.04 | 1.45-1.48 (m, 3H) |
| 7a | 24.93 | 1.84 (d, J = 7.5, 1H) |
| 7b | 24.93 | 1.19-1.35 (m, 8H) |
| 8 | 31.2 | 2.07 (d, J = 4.1, 2H) |
| 9 | 132.55 | |
| 10 | 125.11 | 6.45 (s, 1H) |
| 10a | 33.78 | 3.06 (d, J = 10.7, 1H) |
| 11 | 23.63 | 1.61 (s, 3H) |
| 12 | 27.85 | 1.19-1.35 (m, 8H) |
| 13 | 19.56 | 0.98 (s, 3H) |
| 1′ | 35.29 | 2.33 (t, J = 7.5, 2H) |
| 2′ | 30.71 | 1.45-1.48 (m, 3H) |
| 3′ | 31.35 | 1.19-1.35 (m, 8H) |
| 4′ | 22.44 | 1.19-1.35 (m, 8H) |
| 5′ | 14.36 | 0.85 (t, J = 7.2, 3H) |

### Reference Data for VB302

Through LC-MS along with 1D and 2D NMR, VB302 was confirmed to be the THC monoglycoside with the glucose residue attached via the 1-OH.

LC-MS. [M+H]⁺ (C₂₇H₄₁O₇) Calcd: m/z=477.3 Found: m/z=477.5. [M+NH₄]⁺ (C₂₇H₄₄O₇N) Calcd: m/z=494.5. Found: m/z=494.5. [M+Na]⁺ (C₂₇H₄₀O₇Na) Calcd: m/z=499.5. Found: m/z=499.5. [M−H]⁺ (C₃₃H₃₉O₇) Calcd: m/z=475.3. Found: m/z=475.3. [M+HCOO]⁻ (C281-14100 Calcd: m/z=521.4. Found: m/z=521.4.

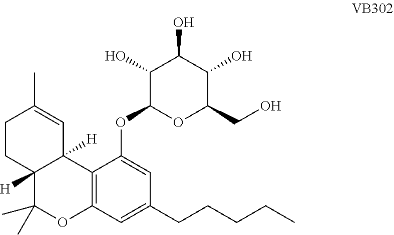 [SMILES: CCCCCc1cc(O[C@@H]2O[C@H](CO)[C@@H](O)[C@H](O)[C@H]2O)c2c(c1)OC(C)(C)[C@@H]1CCC(C)=C[C@@H]21]

TABLE 2
¹H (600 MHz) and ¹³C (150 MHz) NMR spectroscopic data
of VB302 (solvent: DMSO-d6).
VB302 (THC monoside)

| Position | δ_{C} | Δ_{H} (J in Hz) | Position | δ_{C} | Δ_{H} (J in Hz) |
|---|---|---|---|---|---|
| 1 | 156.78 | | Glucose Signals | | |
| 1a | 111.36 | | 1″ | 100.75 | 4.84 (d, J = 7.0, 1H, H1β) |
| 1-OH | | | 2″ | 70.38 | 3.17-3.30 (m, 5H) |
| 2 | 106.57 | 6.46 (s, 1H) | 3″ | 77.48 | 3.17-3.30 (m, 5H) |
| 3 | 142.12 | | 4″ | 74.05 | 3.17-3.30 (m, 5H) |
| 4 | 110.87 | 6.21 (s, 1H) | 5″ | 77.62 | 3.17-3.30 (m, 5H) |
| 5 | 154.08 | | 6″a | 61.23 | 3.71 (m, 1H) |
| 6 | 77.09 | | 6″b | | 3.47 (m, 1H) |
| 6a | 46.14 | 1.48-1.55 (m, 3H) | 3-OH | | 4.53 (t, J = 5.6, 1H) |
| 7a | 24.95 | 1.87 (dd, J = 2.3, 7.6, 1H) | 4-OH | | 4.96 (dd, J = 5.0, 1H) |
| 7b | | 1.21-1.36 (m, 8H) | 5-OH | | 5.00 (d, J = 2.1, 1H) |
| 8 | 31.21 | | | | |
| 9 | 132.43 | | | | |
| 10 | 125.92 | 6.33 (s, 1H) | | | |
| 10a | 33.72 | 3.17-3.30 (m, 5H) | | | |
| 11 | 23.66 | 1.61 (s, 3H) | | | |
| 12 | 27.97 | 1.21-1.36 (m, 8H) | | | |
| 13 | 19.5 | 0.99 (s, 3H) | | | |
| 1′ | 35.52 | 2.41 (t, J = 7.5, 2H) | | | |
| 2′ | 30.58 | 1.48-1.55 (m, 3H) | | | |
| 3′ | 31.34 | 1.21-1.36 (m, 8H) | | | |
| 4′ | 22.41 | 1.21-1.36 (m, 8H) | | | |
| 5′ | 14.35 | 0.85 (t, J = 7.2, 3H) | | | |

Characterization of VB309

Through LC-MS along with 1D and 2D NMR, VB309 was determined to be a linear THC diglycoside. The anomeric carbon of the primary glucose is bound to the THC aglycone via the 1-OH group of THC. The secondary linear glucose residue is attached to the primary glucose by β-1-4-glycosidic linkage.

LC-MS. [M+H]⁺ (C33H₅₁O₁₂) Calcd: m/z=639.5. Found: m/z=639.5. [M+NH₄]⁺ (C₃₃H₅₄O₁₂N) Calcd: m/z=656.5. Found: m/z=656.5. [M+Na]⁺ (C₃₃H₅₀O₁₂Na) Calcd: m/z=661.5. Found: m/z=661.5. [M−H]⁻ (C₃₃H₄₉O₁₂) Calcd: m/z=637.5. Found: m/z=637.6. [M+HCOO]⁻ (C₃₄H₅₁O₁₄) Calcd: m/z=683.3. Found: m/z=683.6.

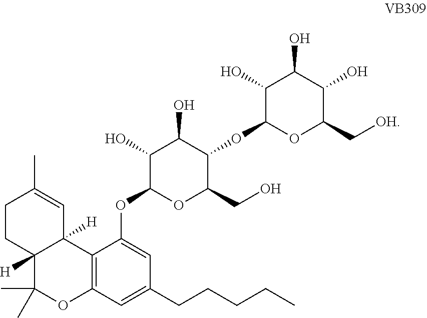 [SMILES: CCCCCc1cc(O[C@@H]2O[C@H](CO)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]

TABLE 3
¹H (600 MHz) and ¹³C (150 MHz) NMR spectroscopic data of VB309
(solvent: DMSO-d6). Chemical shifts based on HSQC, HMBC and H2BC spectrum.
VB309 (THC diglycoside, beta-1-4 linkage)

| Position | δ_{C} | Δ_{H} (J in Hz) | Position | δ_{C} | Δ_{H} (J in Hz) |
|---|---|---|---|---|---|
| 1 | 156.58 | | Glucose 1 Signals | | |
| 1a | 111.35 | | 1″ | 100.21 | 5.25 (d, J = 5.9, 1H) |
| 1-OH | | | 2″ | 73.87 | 3.36-3.56 (m, 5H) |
| 2 | 106.54 | 6.45 (s, 1H) | 3″ | 76.96 | 3.07-3.25 (m, 3H) |
| 3 | 142.13 | | 4″ | 77.4 | 3.36-3.56 (m, 5H) |
| 4 | 110.95 | 6.21 (s, 1H) | 5″ | 75.53 | 3.36-3.56 (m, 5H) |
| 5 | 154.15 | | 6″a | 60.62 | 3.65-3.77 (m, 3H) |
| 6 | 77.11 | | 6″b | | 3.65-3.77 (m, 3H) |
| 6a | 46.13 | 1.48-1.55 (m, 3H) | Glucose 2 Signals | | |
| 7a | 24.93 | 1.87 (d, J = 9.9, 1H) | 1′′′ | 103.67 | 4.32 (d, J = 7.8, 1H) |
| 7b | | 1.24-1.41 (m, 8H) | 2′′′ | 73.87 | 2.77-2.95 (m, 2H) |
| 8 | 31.2 | 2.08 (d, J = 4.8, 2H) | 3′′′ | 77.32 | 3.07-3.25 (m, 3H) |
| 9 | 132.57 | | 4′′′ | 70.51 | 2.77-2.95 (m, 2H) |
| 10 | 125.88 | 6.32 (s, 1H) | 5′′′ | 75.75 | 3.36-3.56 (m, 5H) |
| 10a | 33.73 | 3.07-3.25 (m, 3H) | 6′′′a | 61.51 | 3.65-3.77 (m, 3H) |
| 11 | 23.67 | 1.61 (s, 3H) | 6′′′b | | 3.36-3.56 (m, 5H) |
| 12 | 27.79 | 1.24-1.41. (m, 8H) | | | |
| 13 | 19.5 | 1.05 (s, 3H) | | | |
| 1′ | 35.5 | 2.42 (t, J = 7.5, 2H) | | | |
| 2′ | 30.39 | 1.48-1.55 (m, 3H) | | | |
| 3′ | 31.3 | 1.24-1.41 (m, 8H) | | | |
| 4′ | 22.4 | 1.24-1.41 (m, 8H) | | | |
| 5′ | 14.35 | 0.85 (t, J = 7.2, 3H) | | | |

Characterization of VB310

Through LC-MS along with 1D and 2D NMR, VB310 was determined to be a linear THC diglycoside. The anomeric carbon of the primary glucose is bound to the THC aglycone via the 1-OH group of THC. The secondary linear glucose residue is attached to the primary glucose by β-1-6-glycosidic linkage.

LC-MS. [M+H]⁺ (C₃₃H₅₁O₁₂) Calcd: m/z=639.5. Found: m/z=639.5. [M+NH₄]⁺ (C₃₃H₅₄O₁₂N) Calcd: m/z=656.5. Found: m/z=656.5. [M+Na]⁺ (C₃₃H₅₀O₁₂Na) Calcd: m/z=661.5. Found: m/z=661.5. [M−H]⁻ (C₃₃H₄₉O₁₂) Calcd: m/z=637.5. Found: m/z=637.6. [M+HCOO]⁻ (C₃₄H₅₁O₁₄) Calcd: m/z=683.3. Found: m/z=683.6.

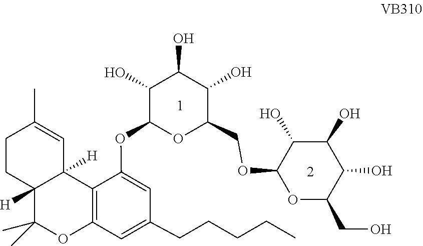 [SMILES: CCCCCc1cc(O[C@@H]2O[C@H](CO[C@@H]3O[C@H](CO)[C@@H](O)[C@H](O)[C@H]3O)[C@@H](O)[C@H](O)[C@H]2O)c2c(c1)OC(C)(C)[C@@H]1CCC(C)=C[C@@H]21]

TABLE 4
¹H (600 MHz) and ¹³C (150 MHz) NMR spectroscopic data of VB310
(solvent: DMSO-d6). Chemical shifts based on HSQC. HMBC and H2BC spectrum.
VB310 (THC diglycoside β-1-6 linkage)

| Position | δ_{C} | Δ_{H} (J in Hz) | Position | δ_{C} | Δ_{H} (J in Hz) |
|---|---|---|---|---|---|
| 1 | 156.81 | | Glucose 1 Signals | | |
| 1a | 111.34 | | 1″ | 104.22 | 4.85 (d, J = 6.9, 1H) |
| 1-OH | | | 2″ | 74.07 | 2.97-3.27 (m, 8H) |
| 2 | 106.79 | 6.51 (s, 1H) | 3″ | 77.44 | 3.41-3.67 (m, 4H) |
| 3 | 142.33 | | 4″ | 70.65 | 2.97-3.27 (m, 8H) |
| 4 | 110.82 | 6.20 (s, 1H) | 5″ | 77.07 | 2.97-3.27 (m, 8H) |
| 5 | 154.01 | | 6″a | 70.29 | 4.00 (d, J = 10.9, 1H) |
| 6 | 77.08 | | 6″b | | 3.41-3.67 (m, 4H) |
| 6a | 46.15 | 1.53 (m, 3H) | Glucose 2 Signals | | |
| 7a | 25.37 | 1.87 (dd, J = 2.7, 9.9, 1H) | 1′′′ | 100.8 | 4.19 (d, J = 7.8, 1H) |
| 7b | 25.37 | 1.24-1.33 (m, 8H) | 2′′′ | 74.07 | 2.97-3.27 (m, 8H) |
| 8 | 31.22 | 2.08 (d, J = 4.4, 2H) | 3′′′ | 77.31 | 2.97-3.27 (m, 8H) |
| 9 | 132.48 | | 4′′′ | 70.45 | 2.97-3.27 (m, 8H) |
| 10 | 125.91 | 6.30 (s, 1H) | 5′′′ | 77.07 | 2.97-3.27 (m, 8H) |
| 10a | 33.71 | 2.97-3.27 (m, 8H) | 6′′′a | 61.61 | 3.41-3.67 (m, 4H) |
| 11 | 23.72 | 1.61 (s, 3H) | 6′′′b | | 3.41-3.67 (m, 4H) |
| 12 | 27.8 | 1.24-1.33 (m, 8H) | | | |
| 13 | 19.51 | 0.98 (s, 3H) | | | |
| 1′ | 35.47 | 2.43 (t, J = 7.5 2H) | | | |
| 2′ | 30.76 | 1.53 (m, 3H) | | | |
| 3′ | 31.38 | 1.24-1.33 (m, 8H) | | | |
| 4′ | 22.45 | 1.24-1.33 (m, 8H) | | | |
| 5′ | 14.41 | 0.86 (t, J = 7.2, 3H) | | | |

Characterization of VB311

Through LC-MS along with 1D and 2D NMR, VB311 was determined to be a branched THC triglycoside. The anomeric carbon of the primary glucose is bound to the THC aglycone via the 1-OH group of THC. The branched glucose residues are attached to the primary glucose by β-1-4-glycosidic and β-1-6-glycosidic linkages.

LC-MS. [M+H]⁺ (C39H₆₁O₁₇) Calcd: m/z=801.5. Found: m/z=801.5. [M+NH₄]⁺ (C₃₉H64017N) Calcd: m/z=818.5. Found: m/z=818.5. [M+Na]⁺ (C39^{-1600,7}Na) Calcd: m/z=823.3. Found: m/z=823.5. [M−H]⁻ (C₃₉H₅₉O₁₇) Calcd: m/z=799.4. Found: m/z=799.6. [M+HCOO]⁻ (C₄₀H₆₁O₁₉) Calcd: m/z=845.4. Found: m/z=845.7.

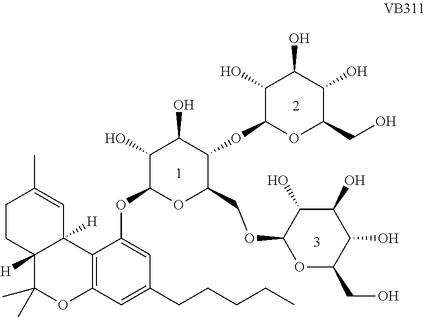 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]

TABLE 5
¹H(600 MHz) and ¹³C (150 MHz) NMR spectroscopic data of VB311
(solvent: DMSO-d6). Chemical shifts based on HSQC, HMBC and H2BC spectrum.
VB311 (THC triglycoside, branched β-1-4, β-1-6 linkage)

| Position | δ_{C} | Δ_{H} (J in Hz) | Position | δ_{C} | Δ_{H} (J in Hz) |
|---|---|---|---|---|---|
| 1 | 156.47 | | Glucose 1 Signals | | |
| 1a | 111.42 | | 1″ | 100.04 | 5.02 (d, J = 7.8, 1H) |
| 1-OH | | | 2″ | 73.97 | 3.63-3.70 (m, 4H) |
| 2 | 106.57 | 6.47 (s, 1H) | 3″ | 79.74 | 3.63-3.70 (m, 4H) |
| 3 | 142.21 | | 4″ | 70.53 | 2.97-3.40 (m, 9H) |
| 4 | 110.93 | 6.22 (s, 1H) | 5″ | 77.11 | 3.40-3.46 (m, 3H) |
| 5 | 154.18 | | 6″a | 68.54 | 4.06 (d, J = 10.4, 1H) |
| 6 | 77.13 | | 6″b | | 3.87 (dd, J = 3.2, 7.8, 1H) |
| 6a | 46.14 | 1.47-1.52 (m, 3H) | Glucose 2 Sighals | | |
| 7a | 24.93 | 1.85 (d, J = 9.6, 1H) | 1′′′ | 103.49 | 4.51 (d, J = 7.9, 1H) |
| 7b | 24.93 | 1.23-1.34 (m, 8H) | 2′′′ | 73.85 | 2.97-3.40 (m, 9H) |
| 8 | 31.22 | 2.07 (d, J = 3.0, 2H) | 3′′′ | 77.35 | 2.97-3.40 (m. 9H) |
| 9 | 132.66 | | 4′′′ | 70.53 | 2.97-3.40 (m, 9H) |
| 10 | 125.81 | 6.30 (s, 1H) | 5′′′ | 77.11 | 2.97-3.40 (m, 9H) |
| 10a | 33.69 | 2.97-3.40 (m, 9H) | 6′′′a | 61.55 | 3.63-3.70 (m, 4H) |
| 11 | 23.74 | 1.61 (s, 3H) | 6′′′b | | 3.40-3.46 (m, 3H) |
| 12 | 27.79 | 1.23-1.34 (m, 8H) | Glucose 3 Signals | | |
| 13 | 19.48 | 0.98 (s, 3H) | 1* | 103.97 | 4.25 (d, J = 7.9, 1H) |
| 1′ | 35.47 | 2.43 (t, J = 7.6 2H) | 2* | 73.69 | 2.97-3.40 (m, 9H) |
| 2′ | 30.7 | 1.47-1.52 (m, 3H) | 3* | 77.25 | 2.97-3.40 (m, 9H) |
| 3′ | 31.34 | 1.23-1.34 (m, 8H) | 4* | 70.46 | 2.97-3.40 (m, 9H) |
| 4′ | 22.44 | 1.23-1.34 (m, 8H) | 5* | 76.99 | 2.97-3.40 (m, 9H) |
| 5′ | 14.38 | 0.86 (t, J = 7.2, 3H) | 6*a | 61.55 | 3.63-3.70 (m, 4H) |
| | | | 6*b | | 3.40-3.46 (m, 3H) |

Characterization of VB312

Through LC-MS along with 1D and 2D NMR, VB312 was determined to be a linear THC triglycoside. The anomeric carbon of the primary glucose is bound to the THC aglycone via the 1-OH group of THC. The secondary linear glucose residue is attached to the primary glucose by β-1-4-glycosidic linkage. The tertiary linear glucose residue is attached to the secondary glucose by β-1-3-glycosidic linkage.

LC-MS. [M+H]⁺ (C₃₉H₆₁O₁₇) Calcd: m/z=801.5. Found: m/z=801.6. [M+NH₄]⁺ (C₃₉H₆₄O₁₇N) Calcd: m/z=818.5. Found: m/z=818.6. [M+Na]⁺ (C₃₉H60O₁₇Na) Calcd: m/z=823.5. Found: m/z=823.6. [M−H]⁻ (C₃₉H₅₉O₁₇) Calcd: m/z=799.4. Found: m/z=799.6. [M+HCOO]⁻ (C₄₀H₆₁O₁₉) Calcd: m/z=845.4. Found: m/z=845.6.

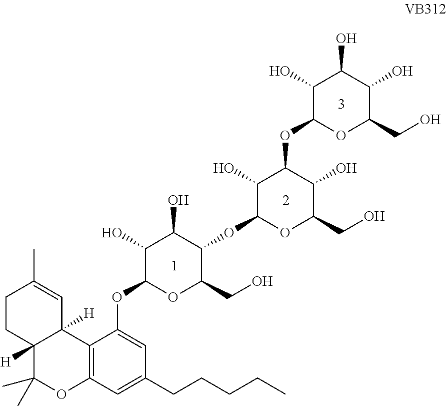 [SMILES: CCCCCc1cc(O[C@@H]2O[C@H](CO)[C@@H](O[C@@H]3O[C@H](CO)[C@@H](O)[C@H](O[C@@H]4O[C@H](CO)[C@@H](O)[C@H](O)[C@H]4O)[C@H]3O)[C@H](O)[C@H]2O)c2c(c1)OC(C)(C)[C@@H]1CCC(C)=C[C@@H]21]

TABLE 6
¹H (600 MHz) and ¹³C (150 MHz) NMR spectroscopic data of VB312
(solvent: DMSO-d6). Chemical shifts based on HSQC, HMBC and H2BC spectrum.
VB312 (THC triglycoside, β-1-4, β-1-3 linkage)

| Position | δ_{C} | Δ_{H} (J in Hz) | Position | δ_{C} | Δ_{H} (J in Hz) |
|---|---|---|---|---|---|
| 1 | 156.57 | | Glucose 1 Signals | | |
| 1a | 111.31 | | 1″ | 100.16 | 4.94 (d, J = 7.8, 1H) |
| 1-OH | | | 2″ | 72.96 | 3.34-3.51 (m, 9H) |
| 2 | 106.54 | 6.46 (s, 1H) | 3″ | 76.78 | 3.17-3.27 (m, 6H) |
| 3 | 142.14 | | 4″ | 80.8 | 3.34-3.51 (m, 9H) |
| 4 | 110.91 | 6.21 (s, 1H) | 5″ | 75.52 | 3.34-3.51 (m, 9H) |
| 6 | 164.14 | | 6″a | 60.59 | 3.(35-3.79 (m, 4H) |
| 6 | 77.12 | | 6″b | | 3.65-3.79 (m, 4H) |
| 6a | 46.13 | 1.48-1.55 (m, 3H) | Glucose 2 Signals | | |
| 7a | 24.93 | 1.87 (d, J = 9.9, 1H) | 1′′′ | 103.06 | 4.44 (d, J = 7.8, 1H) |
| 7b | | 1.23-1.34 (m, 8H) | 2′′′ | 68.92 | 3.17-3.27 (m, 6H) |
| 8 | 31.2 | 2.08 (d, J = 3.9, 2H) | 3′′′ | 88.22 | 3.34-3.51 (m, 9H) |
| 9 | 132.51 | | 4′′′ | 73.76 | 3.34-3.51 (m, 9H) |
| 10 | 125.8 | 6.32 (s, 1H) | 5′′′ | 75.73 | 3.34-3.51 (m, 9H) |
| 10a | 33.73 | 3.17-3.27 (m, 6H) | 6′′′a | 61.33 | 3.65-3.79 (m, 4H) |
| 11 | 23.67 | 1.61 (s, 3H) | 6′′′b | | 3.34-3.51 (m, 9H) |
| 12 | 27.79 | 1.23-1.34 (m, 8H) | Glucose 3 Signals | | |
| 13 | 19.5 | 0.98 (s, 3H) | 1* | 104.61 | 4.33 (d, J = 7.8, 1H) |
| 1′ | 35.51 | 2.41 (t, J = 7.5, 2H) | 2* | 70.61 | 3.02-3.08 (m, 2H) |
| 2′ | 30.6 | 1.48-1.55 (m, 3H) | 3* | 76.6 | 3.17-3.27 (m, 6H) |
| 3′ | 31.31 | 1.23-1.34 (m, 8H) | 4* | 74.28 | 3.02-3.08 (m, 2H) |
| 4′ | 22.41 | 1.23-1.34 (m, 8H) | 5* | 77.45 | 3.17-3.27 (m, 6H) |
| 5′ | 14.35 | 0.85 (t, J = 7.2, 3H) | 6*a | 61.57 | 3.65-3.79 (m, 4H) |
| | | | 6*b | | 3.34-3.51 (m, 9H) |

Characterization of VB313

Through LC-MS along with 1D and 2D NMR, VB313 was determined to be a branched THC tetraglycoside. The anomeric carbon of the primary glucose is bound to the THC aglycone via the 1-OH group of THC. The branched glucose residues are attached to the primary glucose by β-1-4-glycosidic and β-1-6-glycosidic linkages. The tertiary linear glucose residue is attached to the β-1-4-linked secondary glucose by β-1-3-glycosidic linkage.

LC-MS. [M+H]⁺ (C₄₅H₇₀O₂₂) Calcd: m/z=963.4. Found: m/z=963.7. [M+NH4]⁺ (C₄₅H₇₄O₂₂N) Calcd: m/z=980.5. Found: m/z=980.7. [M+Na]⁺ (C₄₅H₇₀O₂₂Na) Calcd: m/z=985.4. Found: m/z=985.6. [M−H]⁻ (C₄₅H₆₉022) Calcd: m/z=961.4. Found: m/z=961.9. [M+HCOO]⁻ (C₄₆H₇₁O₂₄) Calcd: m/z=1007.4. Found: m/z=1007.9.

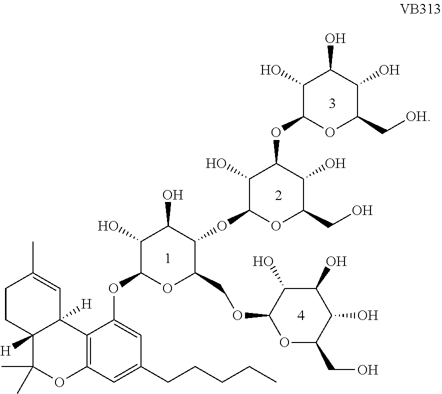 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O[C@@H]4O[C@H](CO)[C@@H](O)[C@H](O)[C@H]4O)[C@H]3O)[C@H](O)[C@H]2O)c2c(c1)OC(C)(C)[C@@H]1CCC(C)=C[C@@H]21]

TABLE 7
¹H (600 MHz) and ¹³C (150 MHz) NMR spectroscopic data of VB313
(solvent: DMSO-d6). Chemical shifts based on HSQC, HMBC and H2BC spectrum.
VB313 (THC tetraglycoside, branched β-1-4-β-1-3, β-1-6 linkage)

| Position | δ_{C} | Δ_{H} (J in Hz) | Position | δ_{C} | Δ_{H} (J in Hz) |
|---|---|---|---|---|---|
| 1 | 156.46 | | Glucose 1 Signals | | |
| 1a | 11.1.4 | | 1″ | 99.97 | 5.00 (d, J = 7.7, 1H) |
| 1-OH | | | 2″ | 73.73 | 3.64-3.77 (m, 4H) |
| 2 | 106.42 | 6.48 (s, 1H) | 3″ | 76.22 | 2.97-3.28 (m, 12H) |
| 3 | 142.22 | | 4″ | 79.81 | 2.97-3.28 (m, 12H) |
| 4 | 110.92 | 6.21 (s, 1H) | 5″ | 77.4 | 3.64-3.77 (m, 4H) |
| 5 | 154.18 | | 6″a | 67.78 | 4.08 (d, J = 10.5, 1H) |
| 6 | 77.12 | | 6″b | | 3.87 (d, J = 10.4, 1H) |
| 6a | 46.14 | 1.47-1.54 (m, 3H) | Glucose 2 Signals | | |
| 7a | 24.93 | 1.87 (d, J = 9.6, 1H) | 1′′′ | 102.69 | 4.64 (d, J = 7.8, 1H) |
| 7b | | 1.21-1.34 (m, 9H) | 2′′′ | 73.82 | 2.97-3.28 (m, 12H) |
| 8 | 31.22 | 2.08 (bs, 2H) | 3′′′ | 88.61 | 2.97-3.28 (m, 12H) |
| 9 | 132.66 | | 4′′′ | 76.46 | 2.97-3.28 (m, 12H) |
| 10 | 125.85 | 6.30 (s, 1H) | 5′′′ | 75.51 | 3.36-3.49 (m, 6H) |
| 10a | 33.69 | 2.97-3.28 (m, 12H) | 6′′′a | 61.21 | 3.64-3.77 (m, 4H) |
| 11 | 23.74 | 1.61 (s, 3H) | 6′′′b | | 3.36-3.49 (m, 6H) |
| 12 | 27.79 | 1.21-1.34 (m, 9H) | Glucose 3 Signals | | |
| 13 | 19.48 | 0.98 (s, 3H) | 1* | 104.52 | 4.31 (d, J = 7.8, 1H) |
| 1′ | 35.48 | 2.43 (t, J = 7.5 2H) | 2* | 74.01 | 2.97-3.28 (m, 12H) |
| 2′ | 30.73 | 1.47-1.54 (m, 3H) | 3* | 77.63 | 2.97-3.28 (m, 12H) |
| 3′ | 31.36 | 1.21-1.34 (m, 9H) | 4* | 70.52 | 2.97-3.28 (m, 12H) |
| 4′ | 22.45 | 1.21-1.34 (m, 9H) | 5* | 68.87 | 3.36-3.49 (m, 6H) |
| 5′ | 14.38 | 0.86 (t, J = 7.2, 3H) | 6*a | 61.57 | 3.64-3.77 (m, 4H) |
| | | | 6*b | | 3.36-3.49 (m, 6H) |
| | | | Glucose 4 Signals | | |
| | | | 1** | 104.08 | 4.24 (d, J = 7.8, 1H) |
| | | | 2** | 74.29 | 2.97-3.28 (m, 12H) |
| | | | 3** | 77.75 | 2.97-3.28 (m, 12H) |
| | | | 4** | 70.62 | 2.97-3.28 (m, 12H) |
| | | | 5** | 72.54 | 2.97-3.28 (m, 12H) |
| | | | 6**a | 61.57 | 3.36-3.49 (m, 6H) |
| | | | 6**b | | 3.36-3.49 (m, 6H) |

Characterization of VB135

Through LC-MS along with 1D and 2D NMR, VB135 was determined to be a branched CBD triglycoside. The anomeric carbon of the primary glucose is bound to the CBD aglycone via the 2′-OH group of CBD. The branched glucose residues are attached to the primary glucose by β-1-3-glycosidic and β-1-4-glycosidic linkages.

LC-MS. [M+H]⁺ (C₃₉H₆₁O₁₇) Calcd: m/z=801.5. Found: m/z=801.6. [M+NH₄]⁺ (C₃₉H₆₄O₁₇N) Calcd: m/z=818.5. Found: m/z=818.7. [M+Na]⁺ (C₃₉H₆₀O₁₇Na) Calcd: m/z=823.5. Found: m/z=823.6. [M−H]⁻ (C₃₉H₅₉O₁₇) Calcd: m/z=799.6. Found: m/z=799.8. [M+HCOO]⁻ (C₄₀H₆₁O₁₉) Calcd: m/z=845.6. Found: m/z=845.8.

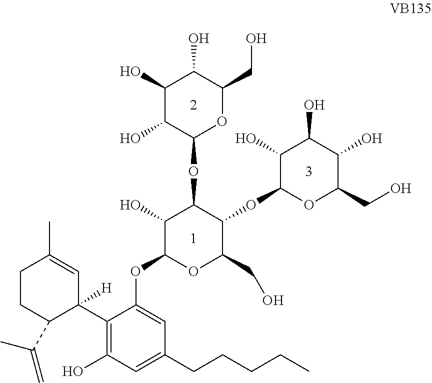

TABLE 8
¹H (600 MHz) and ¹³C (150 MHz) NMR spectroscopic data of VB135
(solvent: DMSO-d6). Chemical shifts based on HSQC, HMBC and H2BC spectrum.
VB135 (CBD triglycoside β-1-3, β-1-4 linkages)

| Position | δ_{C} | Δ_{H} (J in Hz) | Position | δ_{C} | Δ_{H} (J in Hz) |
|---|---|---|---|---|---|
| 1 | 36.11 | 4.95 (bs, 1H) | Glucose 1 Signals | | |
| 2 | 127.11 | 5.09 (s, 2H) | 1′′′ | 100.55 | 4.85 (d, J = 7.7, 1H) |
| 3 | 131.03 | | 2′′′ | 73.73 | 3.08-3.20 (m, 9H) |
| 4 | 30.8 | | 3′′′ | 81.41 | 3.87 (t, J = 8.7, 1H) |
| 4a | | 2.10 (m, 1H) | 4′′′ | 76.71 | 3.08-3.20 (m, 9H) |
| 4b | | 1.92 (m, 1H) | 5′′′ | 74.14 | 3.44-3.53 (m, 4H) |
| 5 | 29.79 | | 6a′′′ | 60.58 | 3.66-3.77 (m, 5H) |
| 5a | | 1.52-1.66 (m, 8H) | 6b′′′ | | 3.66-3.77 (m, 5H) |
| 5b | | 1.52-1.66 (m, 8H) | Glucose 2 Signals | | |
| 6 | 44.01 | 3.32 (under H20 pk, 1H) | 1* | 102.8 | 4.71 (d, J = 7.8, 1H) |
| 7 | 23.73 | 1.52-1.66 (m, 8H) | 2* | 70.21 | 3.08-3.20 (m, 9H) |
| 8 | 44.01 | | 3* | 76.98 | 3.08-3.20 (m, 9H) |
| 9 | 110.37 | | 4* | 73.01 | 3.66-3.77 (m, 5H) |
| 9 cis | | 4.37 (s, 1H) | 5* | 76.16 | 3.44-3.53 (m, 4H) |
| 9 trans | | 4.52 (m, 2H) | 6a* | 61.41 | 3.66-3.77 (m, 5H) |
| 10 | 19.81 | 1.52-1.66 (m, 8H) | 6b* | | 3.44-3.53 (m, 4H) |
| 1′ | 117.46 | | Glucose 3 Signals | | |
| 2′ | 149.36 | | 1** | 101.1 | 4.52 (d, J = 7.7, 1H) |
| 3′ | 106.77 | 6.33 (s, 1H) | 2** | 70.29 | 3.08-3.20 (m. 9H) |
| 4′ | 141.01 | | 3** | 74.37 | 3.08-3.20 (m, 9H) |
| 5′ | 110.37 | 6.23 (s, 1H) | 4** | 76.77 | 3.08-3.20 (m, 9H) |
| 6′ | 156.78 | | 5** | 77.53 | 3.08-3.20 (m, 9H) |
| 1″ | 35.56 | 2.37 (t, J = 7.5, 2H) | 6a** | 61.52 | 3.66-3.77 (m, 5H) |
| 2″ | 30.61 | 1.49 (t, J = 7.2, 2H) | 6b** | | 3.44-3.53 (m, 4H) |
| 3″ | 22.43 | 1.24-1.31 (m, 4H) | | | |
| 4″ | 31.44 | 1.24-1.31 (m, 4H) | | | |
| 5″ | 14.37 | 0.86 (t, J-7.1, 9H) | | | |

The above cannabinoid di-, tri- and tetra-glycosides are structurally distinct from anything previously characterized, and the following sections will present the in vitro and in vivo properties that distinguish them from previously known cannabinoid glycosides.

### Human Cannabinoid Receptor Binding Studies

Pharmacology screening was performed with VB302 and VB311 to assess whether THC-glycosides still bound to the cannabinoid receptors. Reference standards for each assay were tested concurrently to ensure accuracy of the individual tests, and Δ9-THC was tested independently to serve as a positive control and reference for the VB302 and VB311 data.

In radioligand binding assays for the human cannabinoid receptors CB1R and CB2R, 10 μM VB302 and VB311 were shown to have significantly reduced binding compared to 10 μM Δ9-THC, with significance defined as greater than 50% inhibition or activation in the assay. The results of the binding assays are summarized in Table 9 (values reported as percent displacement of the binding comparison agent by the test compound).

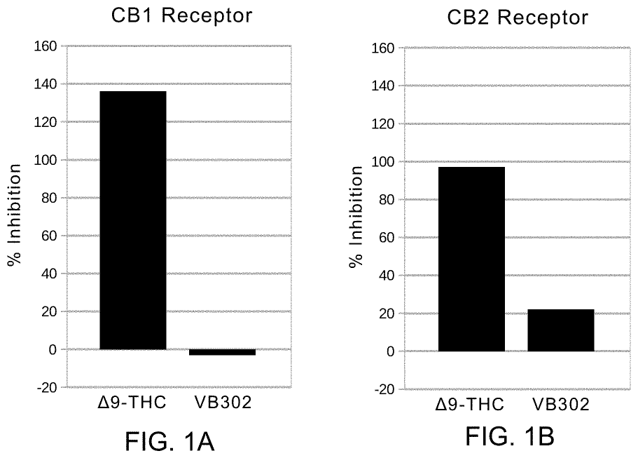

In assays with human CB1R, the comparison agent [³H] SR₁₄₁₇₁₆A (radiolabeled Rimonabant, 2 nm) was displaced 136% by 10 μM Δ9-THC, whereas VB302 and VB311 did not significantly inhibit binding at the receptors (−3% and 3% reported inhibition, respectively). This indicates that the THC-glycoside no longer binds in the active site of the human CB1R. The assay was performed in human recombinant Chem-1 cells, with 2.0 nanomolar [³H] SR₁₄₁₇₁₆A, 90 minutes at 37C in 50 mM HEPES, pH 7.4, 5 mM MgCl₂, 1 mM CaCl₂, 0.2% BSA. The results of the Δ9-THC and VB302 inhibition assay of the human cannabinoid receptor type 1 (CB.1R) are graphically depicted in FIG. 1(a).

Both Δ9-THC and the test compound were tested at a concentration of 10 μM, which was chosen because Δ9-THC has a Kᵢ for CB1 in the range of 5-80 nM, so the relatively high concentration of 10 μM was expected to show displacement at CB1 if the test compounds still bound to the receptor (Kᵢ data from: Pertwee, Roger G. “Pharmacological actions of cannabinoids” in Cannabinoids, pp. 1-51. Springer, Berlin, Heidelberg, 2005.).

Similarly, in assays with human CB2R, the comparison agent [³H] WIN-55,212-2 (radiolabeled CB2R ligand, 2.4 nm) was displaced 97% by 10 μM Δ9-THC, whereas VB302 and VB311 did not significantly inhibit binding at the receptor with ligand displacement values of 17% and 21%, respectively. These results suggest that even the addition of a single glucose moiety to the hydroxyl group of Δ9-THC impairs binding at CB1R, and significantly inhibits binding at CB2R at these supraphysiologic ligand concentrations. The assay was performed in human recombinant CHO-K1 cells, with 2.40 nanomolar [³H] R(+)-WIN-55,212-2, and non-specific ligand was 10.0 micromolar R(+)-WIN-55,212-2, 90 minutes at 37C in 20 mM HEPES, pH 7.0, 0.5% BSA. The results of the Δ9-THC and VB302 inhibition assay of the human cannabinoid receptor type 2 (CB2R) are graphically depicted in FIG. 1(b).

Taken together, these industry standard pharmacology results indicate that VB302 and VB311 and more generally glycosylation of Δ9-THC do not result in substances with binding characteristics consistent with binding at the human CB1 or CB2 receptors.

Due to the similarity between the CB receptor assays, it is likely that any addition of a sugar to the hydroxyl group of THC or other cannabinoids prevents them from binding within the active site of the cannabinoid receptors. This is consistent with the observed lack of psychoactivity of THCA-glycosides and THC-11-OH-glucuronide reported in McPartland et al. 2017.

Table 9 provides a summary of the full safety pharmacology screen results for Δ9-THC, VB302, and VB311. An industry standard pharmacology screen was performed for Δ9-THC, VB302, and VB311. The “Safety Screen 44” was performed at 10 micromolar for each test article against a list of human targets that are predictive of adverse toxicological events in humans (Bowes, J. et al. “Reducing safety-related drug attrition: the use of in vitro pharmacological profiling.” Nature Reviews Drug Discovery 11, no. 12 (2012): 909.). The values for each test article represent the percent inhibition of the listed receptor or transporter, as determined by displacement of the control radiolabeled ligand. Results greater than 50% were deemed significant. Cells that are highlighted signify a “significant” response in the assay. Δ9-THC was found to inhibit or disrupt the binding for 16 of the 44 known pharmacological targets at 10 micromolar test article concentration (see highlighted entries in Table 9), whereas VB302 and VB311 did not significantly alter any of the targets at the same concentration.

TABLE 9
Binding data for safety screen panel of human pharmacological targets

| ASSAY NAME | THC | VB302 | V8311 |
|---|---|---|---|
| 5-HT transporter (h) (antagonist radioligand) | 91 | −1 | 3 |
| 5-HT1A (h) (agonist radioligand) | −27 | 6 | -6 |
| 5-HT1B (antagonist radioligand) | 26 | −1 | −7 |
| 5-HT2A (h) (agonist radioligand) | 92 | 11 | 4 |
| 5-HT2B (h) (agonist radioligand) | 90 | −5 | −7 |
| 5-HT3 (h) (antagonist radioligand) | −21 | −4 | −13 |
| A2A (h) (agonist radioligand) | 63 | −11 | −4 |
| acetylcholinesterase (h) | 30 | −1 | 1 |
| alpha 1A (h) (antagonist radioligand) | 6 | −5 | −3 |
| alpha 2A (h) (antagonist radioligand) | 94 | 0 | 8 |
| AR (h) (agonist radioligand) | 13 | 4 | 10 |
| beta 1 (h) (agonist radioligand) | 1 | 7 | 0 |
| beta 2 (h) (antagonist radioligand) | −12 | 2 | −1 |
| BZD (central) (agonist radioligand) | 2 | −10 | 16 |
| Ca2 + channel (L, dihydropyridine site) (antagonist radioligand) | 59 | 22 | −7 |
| CB1 (h) (agonist radioligand) | 136 | −3 | 3 |
| CB2 (h) (agonist radioligand) | 97 | 22 | 17 |
| CCK1 (CCKA) (h) (agonist radioligand) | 87 | −31 | 17 |
| COX1 (h) | 9 | 30 | 11 |
| COX2 (h) | −6 | −20 | 8 |
| D1 (h) (antagonist radioligand) | 75 | 4 | 37 |
| D2S (h) (agonist radioligand) | 13 | 22 | −6 |
| delta (DOP) (h) (agonist radioligand) | 43 | 5 | 0 |
| dopamine transporter (h) (antagonist radioligand) | 101 | 18 | 28 |
| ETA (h) (agonist radioligand) | 14 | −21 | 9 |
| GR (h) (agonist radioligand) | 20 | −8 | 12 |
| H1 (h) (antagonist radioligand) | 8 | 10 | −7 |
| H2 (h) (antagonist radioligand) | −6 | −11 | −10 |
| kappa (KOP) (agonist radioligand) | 92 | 20 | 4 |
| KV channel (antagonist radioligand) | 5 | 1 | −5 |
| Lck kinase (h) | 15 | −10 | 21 |
| M1 (h) (antagonist radioligand) | 20 | −3 | −8 |
| M2 (h) (antagonist radioligand) | 21 | 2 | −4 |
| M3 (h) (antagonist radioligand) | 23 | 11 | −5 |
| MAO-A (antagonist radioligand) | 1 | 5 | 2 |
| mu (MOP) (h) (agonist radioligand) | 89 | −5 | 5 |
| N neuronal alpha 4beta 2 (h) (agonist radloligand) | 1 | 7 | −1 |
| Na + channel (site 2) (antagonist radioligand) | 65 | 13 | 5 |
| NMDA (antagonist radioligand) | 10 | 8 | 3 |
| norepinephrine transporter (h) (antagonist radioligand) | 90 | 16 | 20 |
| PDE3A (h) | −4 | −44 | −6 |
| PDE4D2 (h) | −11 | −8 | −6 |
| Potassium Channel hCRG (human)-[3ll] Dofetilide | 67 | −23 | −10 |
| Via (h) (agonist radioligand) | −2 | 8 | 7 |

Abbreviations for Table 9: (h) = human. 5-HT transporter = Serotonin (5-Hydroxytryptamine) transporter. 5-HT1A = Serotonin (5-Hydroxytryptamine) 5-HT1A receptor. 5-HT1B = Serotonin (5-Hydroxytryptamine) 5-HT1B receptor. 5-HT2A = Serotonin (5-Hydroxytryptamine) 5-HT2A receptor. 5-HT2B = Serotonin (5-Hydroxytryptamine) 5-HT2B receptor. 5-HT3 = Serotonin (5-Hydroxytryptamine) 5-HT3 channel. A2A = Adenosine A2A receptor. Alpha 1A = Adrenergic α1A receptor. Alpha 2A = Adrenergic α2A receptor. AR = Adrenergic α2A receptor. Beta 1 = Adrenergic β1 receptor. Beta 2 = Adrenergic β2 receptor. BZD (central) = Benzodiazapine GABA channel. Ca2 + channel = Calcium Channel L-Type, Dihydropyridine. CB1 = Cannabinoid 1 receptor. CB2 = Cannabinoid 2 receptor. CCK1 (CCKA) = Cholecystokinin CCK1 (CCKA). COX1 = Cyclooxygenase-1. COX2 = Cyclooxygenase-2. D1 = Dopamine 1 receptor. D2S = Dopamine D2S receptor. Delta (DOP) = Opiate δ1 receptor. ETA = Endothelin ETA receptor. GR = Glucocorticoid receptor. H1 = Histamine 1 receptor. H2 = Histamine 2 receptor. Kappa (KOP) = Opiate κ receptor. KV channel = Voltage-gated potassium channel. Lck kinase = lymphocyte-specific Protein Tyrosine Kinase. M1 = Muscarinic M1 receptor. M2 = Muscarinic M2 receptor. M3 = Muscarinic M3 receptor. MAO-A = Monoamine Oxidase. Mu (MOP) = Opiate μ receptor. N neuronal alpha 4beta 2 = Nicotinic Acetylcholine α4β2. Na + channel (site 2) = Sodium channel. NMDA = N-Methyl-D-aspartate. PDE3A = Phosphodiesterase 3A. PDE4D2 = Phosphodiesterase 4D2. Via = Vasopressin V1A receptor.

It is clear from these results that cannabinoid glycosides including VB302 and VB311 are largely functionally inert at the cannabinoid receptors, and thus must be activated prior to retaining activity in a biological system.

### In Vitro Glycoside Hydrolase Studies

In addition to NMR structural characterization, in vitro enzymatic digestion of THC-glycosides was performed with commercially available glycoside hydrolase enzymes to probe and confirm the structural conformations of the sugars on the THC-glycosides. These studies were carried out using numerous enzymes that have been developed by the biofuels and alcohol production industry for the efficient digestion of carbohydrates, as well as other microbial or human enzymes that are easily obtained. More than 20 enzymes were obtained and initially screened against a mixture of THC-glycosides. If hydrolytic activity was observed, further tests were performed with single glycosides to confirm the specific activity towards sugar linkages.

Multiple glycoside hydrolases were found to digest all secondary sugars from the THC-glycosides, with a majority of enzymes producing the THC-monoglycoside VB302 upon complete digestion.

Cannabinoid-glycosides are decoupled by glycoside hydrolases in vitro. Glycoside hydrolases were obtained from commercial sources and reactions were performed according to their individual recommended reaction conditions.

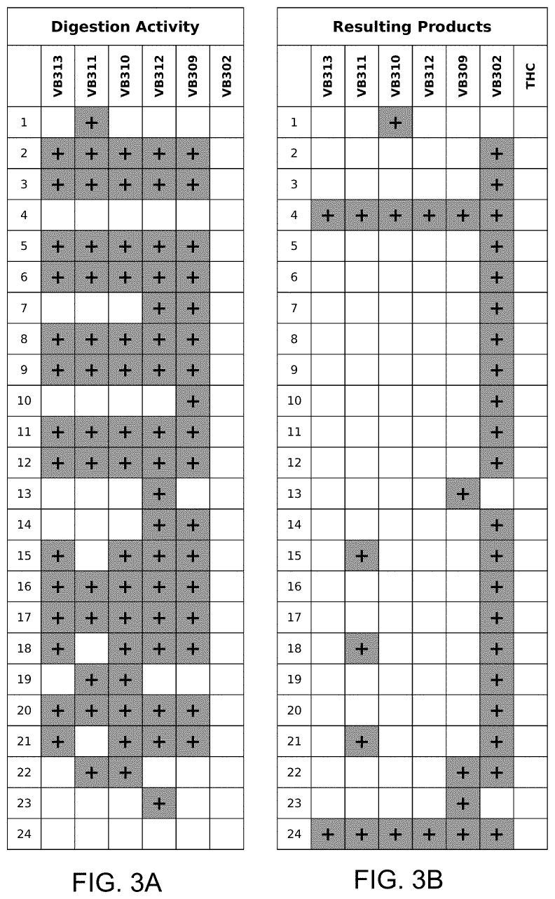

THC-glycosides tested in this assay included VB302, VB309, VB310, VB311, VB312, and VB313. THC-glycosides were initially screened in mixtures, and if activity was observed then follow-up experiments were performed on individual glycosides or narrow mixtures. The results of the digestion assays are summarized in FIG. 3(a). A shaded box with+for Digestion Activity indicates that the particular THC-glycoside is susceptible to degradation by that enzyme. A white/empty box indicates the glycoside displayed no degradation by the respective enzyme. The glycoside hydrolases tested for activity against THC-glycosides are listed in Table 10:

TABLE 10
List of glycoside hydrolases tested for activity against THC-glycosides

| # | Product/Enzyme Name: | Vendor: | Product # | CAS # |
|---|---|---|---|---|
| 1 | Hemicellulase from | Sigma | H2125 | 9025-56-3 |
| | Aspergillis niger | | | |
| | (xylanase/mananase/etc) | | | |
| 2 | Beta-glucosidase from | Gusmer | TS-E | |
| | Almonds | Enterprises | 1984 | |
| | | Inc | | |
| 3 | Cellulase from Trichoderma | Sigma | C2730 | 9012-54-8 |
| | reesei (Celluloclast 1.5 L) | | | |
| 4 | Beta Glucanase from | Sigma | 49101 | 9074-98-0 |
| | Aspergillus niger | | | |
| 5 | Pectinase from | Sigma | 17389 | 9032-75-1 |
| | Aspergillus niger | | | |
| 6 | Endo-1,4-B-D-glucanase from | Sigma | E2164 | 9012-54-8 |
| | Acidothermus cellulolyticus | | | |
| 7 | B-Glucanase from | Sigma | G4423 | 62213-14-3 |
| | Trichoderma | | | |
| | longibrachiatum | | | |
| 8 | Beta Glucosidase from | Creative | NATE- | 9001-22-3 |
| | Aspergillis niger | Enzymes | 1088 | |
| 9 | Driselase Basidiomycetes Sp. | Sigma | D8037 | 85186-71-6 |
| 10 | Chitinase From | Sigma | C6137 | 9001-06-03 |
| | Streptomyces griseus | | | |
| 11 | Cellobiohydrolase I from | Sigma | E6412 | 647-003- |
| | Hypocrea jecorina | | | 00-9 |
| 12 | Lysing Enzymes from | Sigma | L1412 | Mixture |
| | Trichoderma harazanium | | | |
| | (cellulase/chitinase/protease) | | | |
| 13 | Exo-1-3-beta-D-glucanase | Megazymes | EXG5AO | 9073-49-8 |
| | from Aspergillis oryzae | | | |
| 14 | Exo-1-3-beta-D-glucanase | Megazymes | EXBGTV | 9073-49-8 |
| | from Trichoderma virens | | | |
| 15 | Beta-glucosidase from | Sigma | 49290 | 9001-22-3 |
| | Almonds | | | |
| 16 | Beta-glucosidase | Richest | | 9001-22-3 |
| | (unknown source) | Group | | |
| | | LTD | | |
| 17 | Beta-glycosidase from | Quigdao | | 9001-22-3 |
| | Aspergillus niger | Franken | | |
| 18 | Beta-glucosidase from | Toyobo | BGH-201 | 9001-22-3 |
| | Almonds | | | |
| 19 | Cellulase from | Sigma | 22178 | 9012-54-8 |
| | Aspergillus niger | | | |
| 20 | Exo-1-3 Beta-glucanase, | Novozymes | Vinotaste | Mixture |
| | Polygalacturonase | | Pro | |
| 21 | Beta-glucosidase and | Scott | Lallzyme | Mixture |
| | Pectinase | Laboratories | Beta ™ | |
| 22 | Pustulanase | Prokazyme | cel136 | |
| 23 | Recombinant Human | R + D | 5969- | |
| | Cytosolic beta- | Systems | GH-012 | |
| | glucosidase/GBA3 | | | |
| | (Glucosylceramide | | | |
| | hydrolase, liver) | | | |
| 24 | Galactase (Lactaid TM) | Amazon | Lactaid | |

The products of the digestion assays are summarized in FIG. 3(b). The resulting products table indicates which individual THC-glycosides were present when treated with the particular enzyme. A shaded box with+indicates the glycoside was present in the resulting reaction mixture following treatment by the respective enzyme, and a white box indicates that no glycolytic product was observed. The following selected observations were made
Enzyme 1 digests VB311 to V310;
Enzymes 15, 18, and 21 all digest a mixture of THC-glycosides to VB311 and VB302
Enzymes 4 and 24 were not active towards THC-glycosides.
Enzyme 22 was active towards VB311 and VB310, and produced VB309 and VB302.
Enzymes 13 and 23 were active towards VB312 and produced VB309.
All remaining enzymes tested and listed in Table 10, including Enzyme 20, degrade all higher glycosides back to VB302.
None of the enzymes tested were capable of hydrolyzing the primary glucose on THC.

In one study, a mixture of THC-glycosides termed VB300X, containing VB311, VB312, VB309, VB310 and VB313, was digested with Lallzyme Beta™ (Lallemand). The mixture of THC-glycosides VB300X treated with Lallzyme Beta™ produces VB311 and VB302. Nearly all VB313 is degraded to VB311, and VB310, VB312, and VB309 are entirely digested into VB302. The observed persistence of branched glycoside structures like VB311 suggests that the branched glycosides confer resistance to specific glycoside hydrolases because of the steric hindrance of the two adjacent secondary glycosylations.

The reactions were performed as follows: 2 mg/ml VB300X mixture in 30% EtOH in water, 20 mM citrate buffer pH 4.0, and 5 mg/ml Lallzyme Bete™ were brought up to 44° C. while stirring. The reactions progressed and were monitored by HPLC and once at completion the reactions were stopped by the addition of 1M NaOH to increase the reaction mixture pH to 7.0. The reaction mixtures were stripped of VB311 and VB302 by diafiltration. Diafiltration was performed using Spectrum KrosFlo 10K mPES hollow fiber tangential flow filtration (TFF) modules, the size dependent on the total reaction volume, with 30% EtOH as the dilutant. VB311 and VB302 were captured by flowing the hollow fiber module permeate through C₁₈ flash chromatography columns with appropriate binding capacity. The loaded C₁₈ columns were washed and fractionated manually, or by using an InterChim PuriFlash system to obtain pure VB311 and VB302 products.

Reactions were also performed as follows: 3 mg/ml VB300X mixture in 10% DMSO in water, 20 mM citrate buffer pH 4.0, and 5 mg/ml Lallzyme Beta™, and processed as previously described.

Reactions were also performed with the Lallzyme Beta™ enzyme immobilized to a support matrix. The reaction volume was pumped through the enzyme/catalyst reactor until the reaction was deemed to be complete, at which time the reaction volume was able to be directly applied to the C₁₈ flash chromatography columns and processed as previously described.

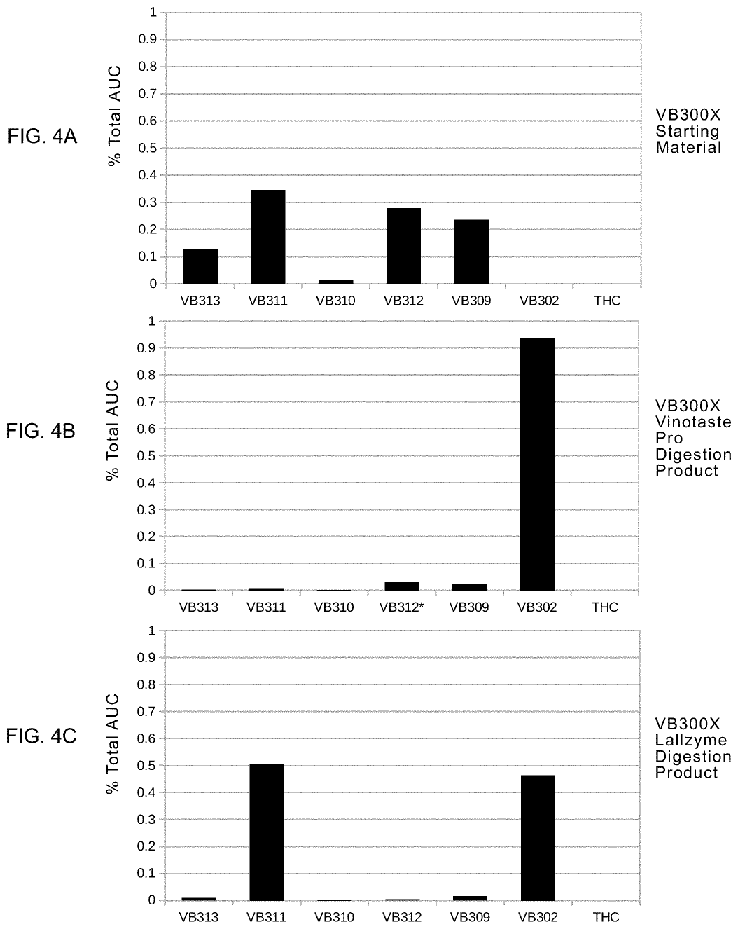

FIG. 4(a) is a graphical depiction of the relative amounts of the starting mixture of THC-glycosides in VB300X as determined by HPLC. Values shown are the percent of the total area under the curve for all THC-glycosides in the mixture. FIG. 4(c) is a graphical depiction of the relative amounts of a final mixture of THC-glycosides following incubation with Lallzyme Beta™ (Lallemand). The starting VB313, VB311, VB310, VB312, and VB309 were largely digested back to VB311 and VB302. It is observed that Lallzyme Beta™ possesses broad glycoside hydrolase activities and is capable of hydrolyzing Beta-1-4 and Beta-1-6 secondary glycosides, but has very low activity towards the branched Beta-1-4 Beta-1-6 triglycoside of THC VB311. No THC was observed at the completion of this reaction.

It has further been observed that, if a mixture of VB300X is left to continue reacting with Lallzyme Beta™ beyond this equilibrium, VB311 will slowly degrade into VB310 and then to VB302, presumably due to weak beta-1-4 glycoside or off target secondary hydrolase activity in the enzymes (results not shown).

In a further study, the THC-glycoside mixture VB300X was digested with Vinotaste Pro (Novozymes). FIG. 4(b) is a graphical depiction of the relative amounts of a final mixture of THC-glycosides following incubation with Vinotaste Pro (Novozymes). The starting VB313, VB311, VB310, VB312, and VB309 were largely digested back to VB302. Vinotaste Pro possesses broad glycoside hydrolase activities and is capable of hydrolyzing Beta-1-4 and Beta-1-6 secondary glycosides, as well as the branched Beta-1-4 Beta-1-6 triglycoside of THC VB311. No THC was observed at the completion of this reaction.

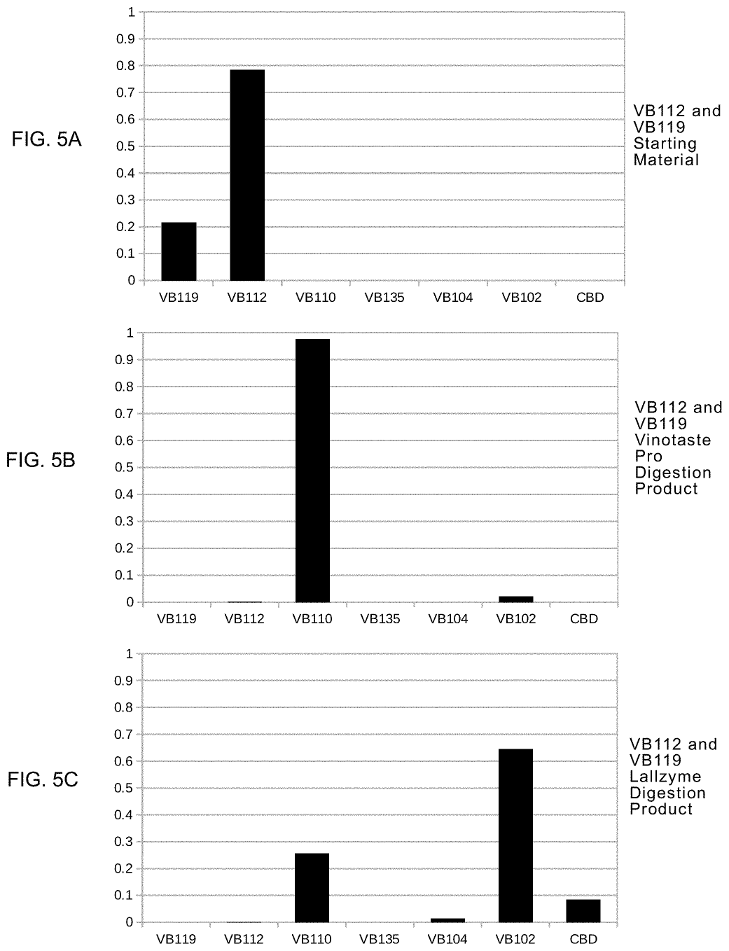

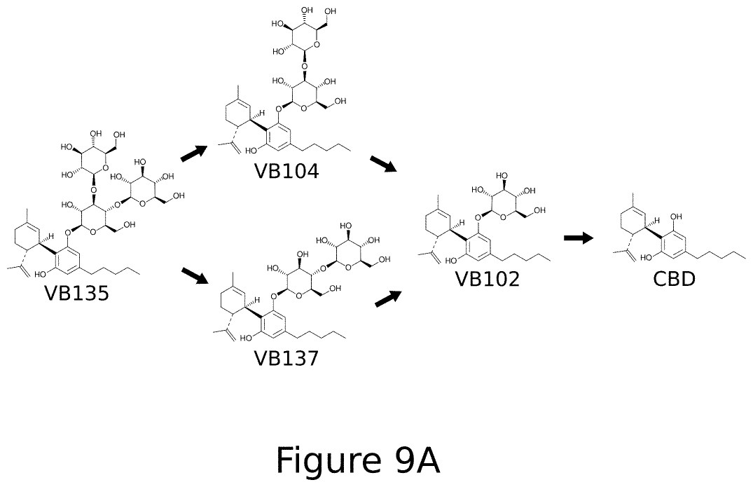

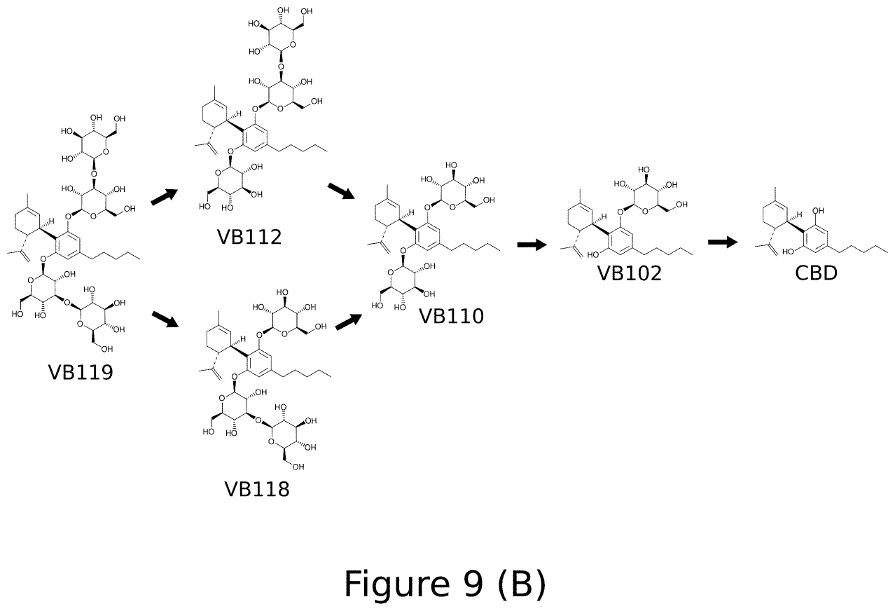

In a further study, a mixture of CBD-glycosides containing VB119 and VB112 were subjected to the same digestion conditions using each of Vinotaste Pro and Lallzyme Beta™ as described above with respect to the VB300X mixture. FIG. 5(a) is a graphical depiction of the relative amounts of the starting mixture of CBD-glycosides containing only VB119 and VB112. See FIG. 9(b) for the proposed decoupling pathways.

FIG. 5(b) is a graphical depiction of the relative amounts of a final mixture of CBD-glycosides following incubation with Vinotaste Pro (Novozymes). The starting VB119 and VB112 were digested back to VB110, with a small amount of VB102 produced in the reaction.

FIG. 5(c) is a graphical depiction of the relative amounts of a final mixture of CBD-glycosides following incubation with Lallzyme Beta™ (Lallemand). The starting VB119 and VB112 were digested back primarily to VB102, with some VB110 and slight CBD present in the product. Lallzyme Beta™ is likely capable of digesting VB119 and VB112 to VB110, but also has activity towards hydrolyzing the primary glucoses from the 2 and 6 hydroxyl groups on the resorcinol ring of CBD, resulting in conversion of VB110 to VB102, and VB102 to CBD. As Lallzyme Beta™ was unable to hydrolyze VB302 back to THC, but capable of hydrolyzing VB110 to VB102 and VB102 to CBD, it is possible that the rotational freedom of CBD is able to conform to the active site of the beta-glucosidase active site present in Lallzyme Beta™.

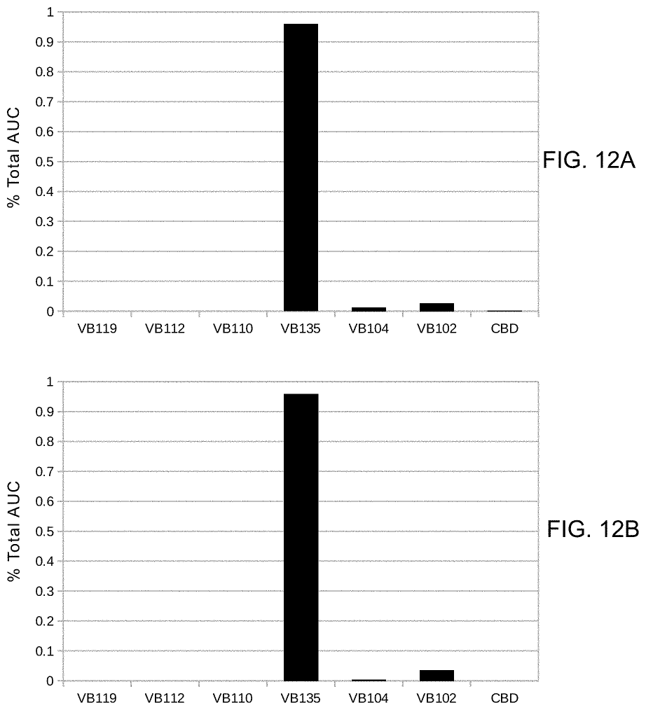

In a further study, VB135 was subjected to the same digestion conditions described above with respect to the VB300X mixture. FIG. 12(a) is a graphical depiction of the relative amounts of a final mixture of CBD-glycosides following incubation of VB135 with Lallzyme Beta™ (Lallemand), and FIG. 12(b) is a graphical depiction of the relative amounts of a final mixture of CBD-glycosides following incubation of VB135 with Vinotaste Pro (Novozymes). VB135 is therefore observed to be highly resistant to hydrolysis by industrial hydrolases such as Vinotaste Pro (Novozymes) and Lallzyme Beta™ (Lallemand). See FIG. 9(a) for the proposed decoupling pathways.

The above hydrolase studies show that, just as glucosyltransferases can carefully build up a dendritic sugar structure via the hydroxyl group on the resorcinol ring of cannabinoids, glycoside hydrolases can carefully break down the glycosylations to produce lower glycosides or even the aglycone base molecules. Hydrolases are also responsible for in vivo decoupling of cannabinoid glycosides inside of the intestinal lumen of animals, highlighting their importance for activation of cannabinoid glycosides.

### Intestinal Absorption Studies

High toxicological doses of THC-glycosides were administered to rats and plasma samples were collected to assess the amount of glycoside, THC aglycone, and metabolites absorbed by the animals.

As VB302 has a higher clogP and is more hydrophobic than higher glycosides such as the tri-glycoside VB311, additional excipients were required for solubilizing in an aqueous mixture at 100 mg/ml for oral gavage in animal studies. Excipients used to prepare the compound solutions for administration by oral gavage were as follows:
VB311: 10% propylene glycol, 10% glycerol, 80% saline
VB302: 20% propylene glycol, 20% glycerol, 10% Tween-20, 50% saline

These excipients were chosen to minimize gastrointestinal effects, but also to minimize solvent assisted cellular uptake.

In one experiment, VB311 was administered by oral gavage at a dosage of 1,000 milligrams per kilogram (mg/kg) to 3 male and 3 female Sprague Dawley rats. Plasma was collected at 1, 2, 6, 24 hours post administration, and THC-glycosides and their metabolites were quantified by extraction using acetonitrile with 0.1% formic acid (v/v) followed by LC-MS analysis. The average maximum concentration (Cₘₐₓ) in the plasma at the time of maximum concentration (Tₘₐₓ) values are listed in Table 11. The area under the curve (AUC) was calculated for each compound and animal and averages are presented.

TABLE 11
Rat plasma toxicokinetic values post VB311 administration at 1,000 mg/kg

| | VB311 | VB310 | VB309 | VB302 | THC | 11-OH-THC |
|---|---|---|---|---|---|---|
| Cₘₐₓ (ng/ml) | 191.0 | 4.4 | 0.8 | 422.1 | 20.0 | 17.3 |
| Tₘₐₓ (hours) | 2.0 | 6.0 | 6.0 | 6.0 | 6.0 | 24.0 |
| AUC (ng/ml*hr) | 2599.8 | 60.1 | 10.4 | 5953.9 | 397.7 | 320.2 |

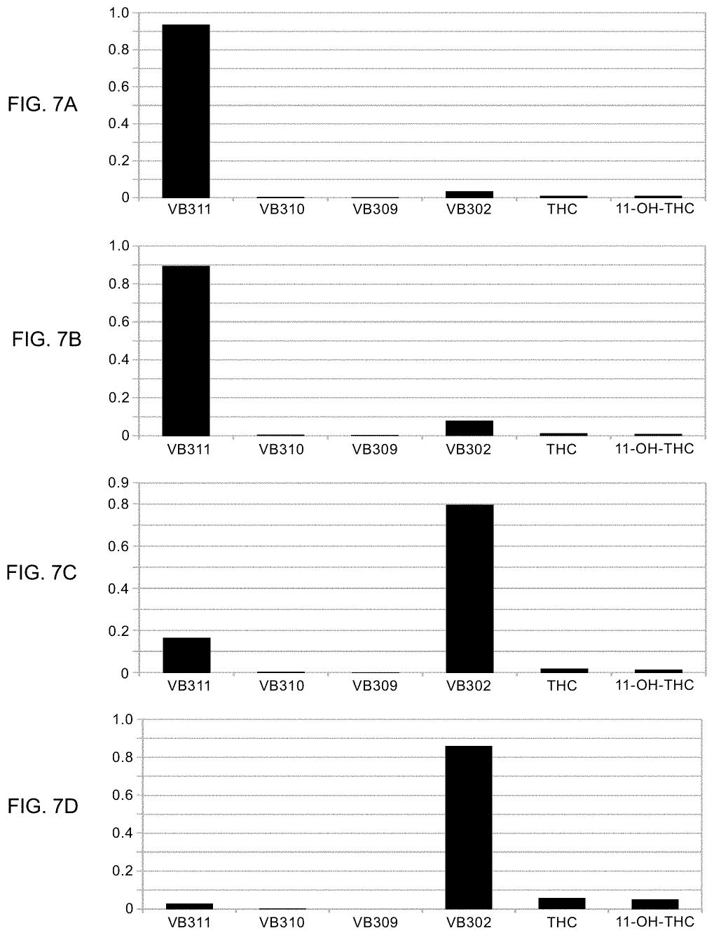

The VB311 and its metabolites were measured at each time point, and are reported in FIGS. 7(a) to 7(d). The measured values are normalized per each timepoint, such that the sum of VB311 and all metabolites at each timepoint=1.

FIG. 7(a) is a graphical depiction of the relative amounts of THC-glycosides present in the plasma of rats 1 hour post oral gavage, and the total quantity of systemic THC-glycosides was low, and VB311 was the relative majority of glycosides present in the plasma at 1 hour. Minor quantities of VB302 were observed.

FIG. 7(b) is a graphical depiction of the relative amounts of THC-glycosides present in the plasma of rats 2 hours post oral gavage, and the total quantity of systemic THC-glycosides was low, and VB311 was the majority of glycosides present in the plasma at 2 hour. Minor quantities of VB302 were observed.

FIG. 7(c) is a graphical depiction of the relative amounts of THC-glycosides present in the plasma of rats 6 hours post oral gavage, and VB311 was extensively modified, and VB302 was the predominant THC-glycoside present in the plasma while THC and the metabolite 11-OH-THC remained at trace levels.

FIG. 7(d) is a graphical depiction of the relative amounts of THC-glycosides present in the plasma of rats 24 hours post oral gavage, and VB311 has been almost completely digested and VB302 relative abundance was greatly increased in the plasma. THC and the metabolite 11-OH-THC increased at 24 hours, but their relative abundance was still low compared to VB302.

This study appears to confirm that activation of the VB311 prodrug is temporally delayed and is based on transit time through the distal small intestine, and activation is fully initiated upon entry into the large intestine.

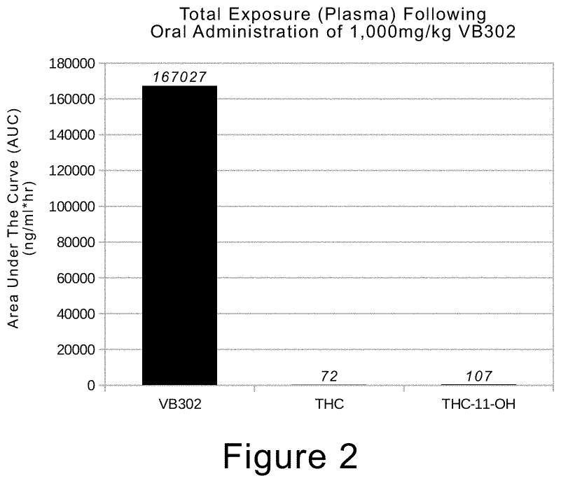

In another experiment, VB302 was administered to Sprague Dawley rats by oral gavage at a dosage of 1,000 milligrams per kilogram (mg/kg) to 3 male and 3 female Sprague Dawley rats. Plasma was collected at 1, 2, 6, 24 hours post administration, and THC-glycosides and their metabolites were quantified by extraction using acetonitrile with 0.1% formic acid (v/v) followed by LC-MS analysis. The average area under the curve (AUC) was calculated for VB302, as well as the intestinal decoupling metabolites Δ9-THC and Δ9-THC-11-OH. Total VB302 plasma AUC was 167,027 ng/ml*hr. Total Δ9-THC plasma AUC was 72 ng/ml*hr. Total Δ9-THC-11-OH plasma AUC was 107 ng/ml*hr. Negligible systemic Δ9-THC was produced in the animals following oral administration of the THC-glycoside VB302. The AUC data for VB302, as well as for the intestinal decoupling metabolites Δ9-THC and Δ9-THC-11-OH, are presented in FIG. 2. This was also observed with other cannabinoid-glycosides, including but not limited to CBD-glycosides.

The high concentration of VB302 in the plasma demonstrates that VB302 has significant absorption and bioavailability, and very little VB302 is decoupled to produce THC. Additionally the low THC concentration suggests that VB302 in the plasma is not decoupled or activated to THC, only in the intestines. The average maximum concentration (Cₘₐₓ) in the plasma at the time of maximum concentration (Tₘₐₓ) values are listed in Table 12.

TABLE 12
Rat plasma toxicokinetic values post
VB302 administration at 1,000 mg/kd

| | VB302 | THC | 11-OH-THC |
|---|---|---|---|
| Cₘₐₓ (ng/ml) | 8339.9 | 4.4 | 7.9 |
| Tₘₐₓ (hours) | 24.0 | 24.0 | 24.0 |
| AUG (ng/ml*hr) | 167027.3 | 72.0 | 106.9 |

VB311 was observed at relatively low levels in the plasma, achieving a Cₘₐₓ of 191.0 ng/ml at the Tₘₐₓ of 2 hours post gavage. Following administration of VB311, intestinal glycosidases likely decoupled the sugars to produce VB302 in the distal ileum and colon, and a Cₘₐₓ for decoupled VB302 in the plasma of 422.1 ng/ml was observed at 6 hours. The 6 hour timepoint correlates with the time required for VB311 to transit to the large intestine and undergo enzyme mediated hydrolysis of the sugars. VB311 decouples to VB302 in the intestines, and due to the increased bioavailability of VB302, the plasma concentration of VB302 is 2.2× higher than VB311 after VB311 administration.

VB302 had significantly higher intestinal absorption compared to VB311, as seen by Cₘₐₓ values of 8,339.9 ng/ml and 191.0 ng/ml, respectively. VB302 is therefore 43x more bioavailable than VB311 after oral administration. VB311 exhibits only 2.3% of the bioavailability of VB302 after oral administration, suggesting that VB302 has higher absorption in the small intestine and upper gastrointestinal tract.

Interestingly, when VB302 was administered directly at 1,000 mg/kg, the amount of systemic THC was far lower than when VB311 was administered at 1,000 mg/kg. Despite VB302 containing 68% more THC by mass than VB311, THC is decoupled and absorbed only 22% as much as VB311 (rats, 4.4 ng/ml plasma THC concentration after VB302, vs 20 ng/ml plasma THC concentration after VB311- both given at 1,000 mg/kg). The result is that VB311 effectively produces 454% more systemic decoupled THC than VB302.

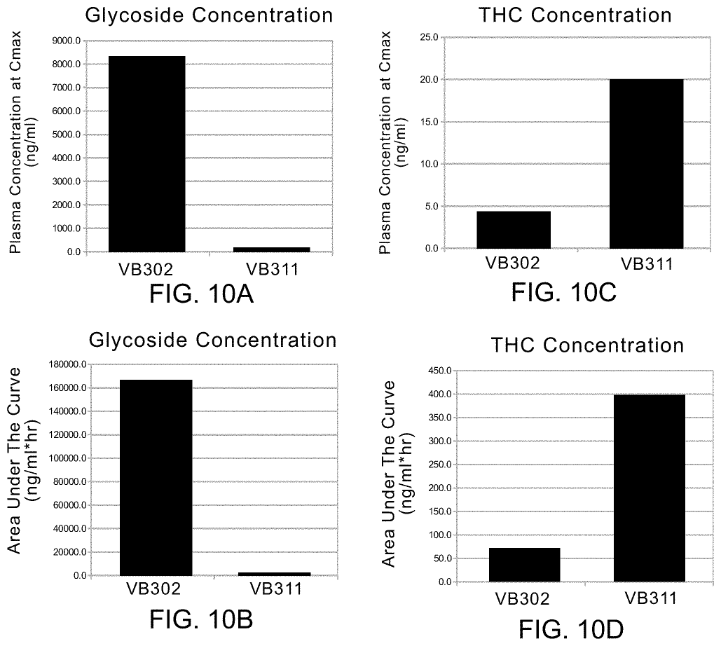

FIG. 10(a) is a graph depicting the plasma Cmax values for VB302 and VB311, following administration of VB302 and VB311, respectively. FIG. 10(b) is a graph depicting the total systemic exposure over 24 hours (AUC) after oral administration of VB302 or VB311. FIG. 10(c) is a graph depicting the plasma Cmax values of decoupled and absorbed THC following administration of VB302 and VB311, respectively. FIG. 10(d) is a graph depicting the total systemic exposure of THC over 24 hours (AUC) after oral administration of VB302 or VB311.

As VB311 appears to be less bioavailable than VB302, less VB311 is observed in the plasma of rats that have been administered VB311. However, because more VB311 stays in the lumen of the gastrointestinal tract, more VB311 reaches the large intestine where glycoside hydrolases are able to activate VB311 to THC. VB311 therefore converts to THC in the large intestine more efficiently than VB302, so less VB311 can be used to deliver similar quantities of THC to the lumen of the large intestine, with much less systemic delivery of THC-glycosides such as VB302.

It should be noted that plasma concentrations of THC are not proportional to the total THC equivalents administered to animals. Rather, THC plasma concentrations are proportional to the amount of THC-glycoside delivered to the large intestine, which in turn is a factor of the specific glycoside composition or structure. Relevant numbers comparing the THC equivalents of VB302 and VB311 are listed in Table 13.

TABLE 13
Comparison of drug composition for
THC-glycosides VB302 and VB311

| | VB302 | VB311 |
|---|---|---|
| Glycoside MW | 476 | 800 |
| THC MW | 314 | 314 |
| % Glucose of Total Molecular Mass | 34.0 | 60.8 |
| % THC of Total Molecular Mass | 66.0 | 39.3 |
| THC mg/kg/Glycoside 1,000 mg/kg | 659.7 | 392.5 |
| THC Equivalents vs VB311 | 1.68 | 1 |
| THC Equivalents vs VB302 | 1 | 0.60 |

Gastrointestinal Tract Decoupling Studies

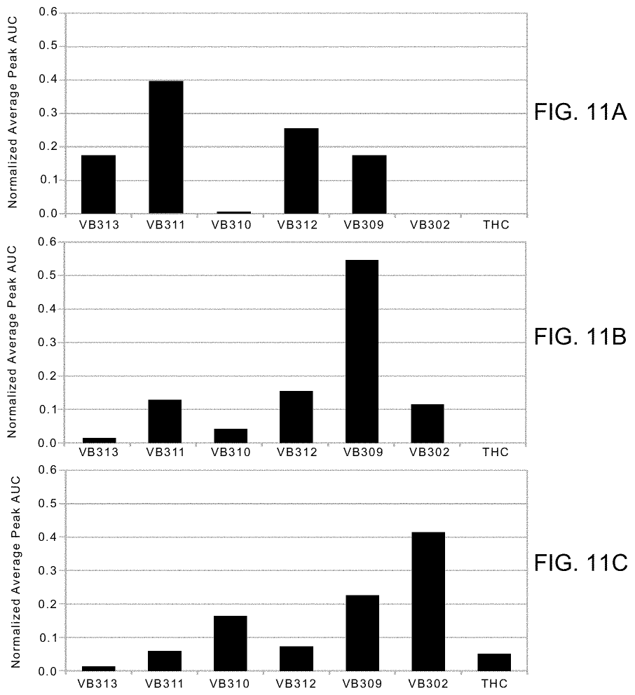

A pharmacokinetic study was carried out in which rats were given 1,000 mg/kg mixed glycosides by oral gavage, the mixture containing VB313, VB311, VB310, VB312 and VB309 in an approximate ratio of 1:2:0.1:1.5:1 (FIG. 11(a)). After 6 hours, the animals were sacrificed and portions of the small and large intestines were collected, snap frozen, and stored at −80 C. Samples were thawed and the intestinal contents were solvent extracted with ethyl acetate (3× equivolume extractions), and the compounds present in the extraction mixture were determined by HPLC to measure the presence of THC-glycosides and their metabolites. The area under the curve for each peak was calculated, and the study group average calculated (only n=2 for 1,000 mg/kg group). The sum for all peaks was determined, and each peak is presented as the percent of total THC-glycosides (normalized as a percentage of the total).

As shown in FIG. 11(b), the extracts from the small intestine (SI) showed a sharp decrease in VB313, VB311, and a modest decrease in VB312, whereas VB309, VB302, and VB310 showed increases. THC was below the limit of detection in the small intestine samples.

As shown in FIG. 11(c), the extracts from the large intestines showed further decreases in VB311, VB312, and a sharp decrease to the VB309 observed in the small intestine extracts. The large intestine samples showed further increases to VB302, VB310, and appreciable amounts of THC.

These studies demonstrate the organ-dependent decoupling or degradation of THC-glycosides following oral administration. Decoupling is first observed in the distal ileum of the small intestine, with a majority of decoupling occurring in the large intestine. THC-glycoside decoupling is dependent on the microbial community in the gastrointestinal tract, specifically on the secreted glycoside hydrolases in the lumen of the gastrointestinal tract.

As the intestinal contents or feces of animals contain a tremendous diversity of microbes, as well as the carbohydrate-digesting enzymes secreted by those bacteria, it would be expected that when THC-glycosides are subjected to the intestines or feces of animals, they would decouple back to THC. The following samples were assayed to determine their respective glycosidic activities on select THC-glycosides:
25=Canus familiarus fecal sample, “Stevie”; Terrier of mixed breed;
26=Canus familiarus fecal sample, “Lucy”; Labrador Retriever;
27=Mus musculus small intestine contents, BALB/C;
28=Mus musculus large intestine contents, BALB/C;
29=Rattus rattus small intestine contents, Sprague Dawley;
30=Rattus rattus large intestine contents, Sprague Dawley;
31=Rattus rattus intestines—inferred from plasma sample, Sprague Dawley;
32=Macaca fascicularis intestines—inferred from plasma sample, Cynomolgus monkey.

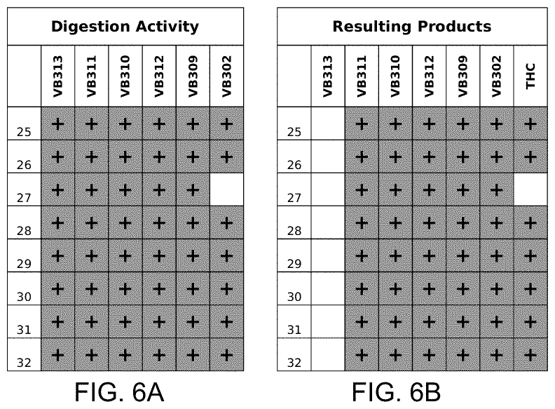

The results of the digestion assays are summarized in FIG. 6(a), in which shaded boxes indicate which samples exhibited carbohydrate-digestion activity. The resulting products of the digestion assays are summarized in FIG. 6(b), in which shaded boxes indicate that the particular compound was formed in the particular matrix.

The intestinal samples 27 to 30 were solvent extracted using 3× equivolume ethyl acetate as previously described, and the compounds present in the extraction mixture were determined by HPLC.

The assay for the Canus familiarus fecal samples was carried out according to the following protocol: A fresh fecal sample was obtained using an ethanol sterilized scoopula and transferred into a sterile 50 ml conical tube. The fecal sample (3 grams) was transferred to a fresh sterile 50 ml conical tube and 30 ml of sterile filtered 1% phosphate buffered saline, pH 7, was added. The fecal sample solution was vortexed to homogenize. Two 2 ml aliquots were removed and filtered using 25 mm 0.45 μm regenerated cellulose (RC) syringe filters to clarify the fecal sample solution.

Cannabinoid glycoside solutions such as VB300X were prepared at 1 mg/ml in deionized water, then sterile filtered through 13 mm 0.2 μm regenerated cellulose (RC) syringe filters.

A series of 1 ml reactions were set up where 500 μl of the glycoside solutions were added to 500 μl of the buffered and clarified fecal sample solution and incubated at 37 ° C. while shaking at 125 rpm for 70 hours.

200 μl samples were pulled and extracted 3 times with 200 μl ethyl acetate (EtOAc) each. The EtOAc was blown off and 200 μl of 50% methanol (MeOH) was added and vortexed to reconstitute. A 1:10 dilution of each was prepared in 50% MeOH and 10 μl injections were analyzcd by HPLC.

If carbohydrate-digestion activity was observed for all constituent VB300X THC-glycosides in the particular matrix, and if THC was observed in the resulting products, then with time all glycosides can be expected to decouple to produce THC. Notably, no THC was observed in the small intestines of mice, suggesting that the glycoside hydrolase capable of removing the primary glucose from VB302 is either absent or expressed at very low levels.

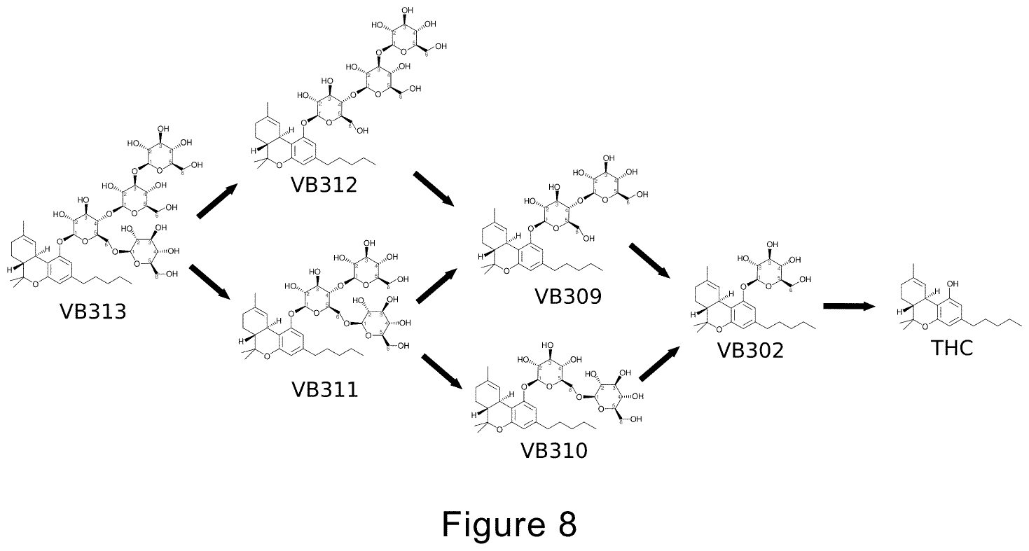

The possible decoupling pathways for the THC-glycosides are shown in FIG. 8. The branched sugars on the THC-glycosides can be removed in differing orders, but both directions ultimately yield the THC-monoglycoside VB302 and then THC. The THC-tetraglycoside VB313 is decoupled to either THC-triglycosides VB311 or VB312. VB312 is then decoupled to the THC-diglycoside V309. VB311 is decoupled to either of the THC-diglycosides VB309 or VB310. VB309 and VB310 are both decoupled to the THC-monoglycoside VB302. VB302 is decoupled to the THC-aglycone.

The possible decoupling pathways for novel and original CBD-glycosides are shown in FIGS. 9(a) and 9(b). Two decoupling pathways exist for CBD-glycosides with glycosylations emanating from either a single hydroxyl group on CBD, or from both hydroxyl groups. The branched CBD-triglycoside VB135 is decoupled to either CBD-diglycoside VB104 or VB137. VB104 and VB137 are both decoupled to the CBD-monoglycoside VB102. VB102 is decoupled to the CBD-aglycone. Separately, the CBD-tetraglycoside VB119 cin be decoupled to either CBD-triglycoside VB112 or VB118. VB112 and VB118 are both decoupled to the CBD-diglycoside VB110.

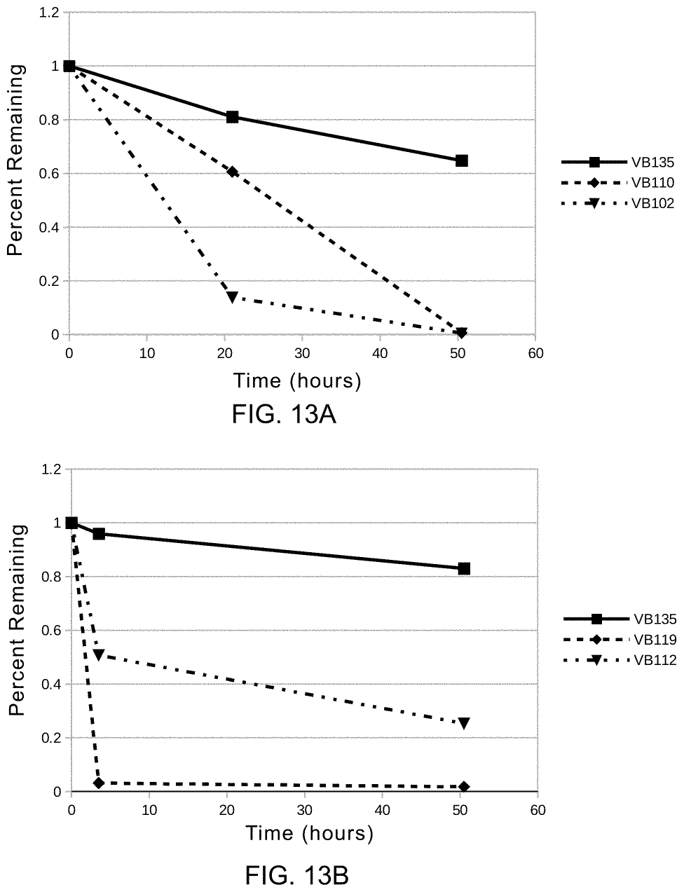

Canine fecal studies were also performed on the CBD-glycosides VB135, VB110 and VB102 using the protocol as previously described, using fecal samples 25 and 26. The results of these studies are reported in FIGS. 13(a) and 13(b).

It was observed in both studies that VB135 exhibited unique resistance to glycoside hydrolase activity in a complex mixture such as canine feces. This recalcitrance greatly exceeds that seen of VB311, the branched triglycoside of THC. Whereas VB311 is branched with β-1-4, and β-1-6 glycoside linkages, VB135 is branched with β-1-3, and β-1-4 glycoside linkages. The relative distance between the branched secondary glycosylations of VB311 is far greater than the distance between secondary glycosylations on VB135. The proximity of the less common β-1-3, and β-1-4 secondary glycoside linkages may contribute to steric hindrance with glycoside hydrolases and may be less compatible with natural glycoside hydrolases.

The novel cannabinoid glycosides described herein have superior bioavailability characteristics over previously characterized glycosides. VB311 exemplifies an ideal cannabinoid glycoside for targeted delivery of THC to the intestinal lumen because it has low systemic absorption, and enhanced release of THC in the lumen of the intestines compared to VB302.

The results of the gastrointestinal tract decoupling studies are consistent with what is known about the relative microbial load distribution in the gastrointestinal tract. Table 14 is a tabular summary of the relative microbial load as defined by organisms per gram of luminal contents at different points along the gastrointestinal tract, including stomach, duodenum, jejunum, proximal ileum, distal ileum and colon (Sartor 2008). The relative distribution of the microbial load correlates to the locations in the GI tract where the THC-glycoside prodrugs appear to be activated, and may explain the observed distal ileum decoupling.

TABLE 14
Relative distribution of microbial
load in gastrointestinal tract

| | Relative |
|---|---|
| | Microbial |
| Organ | Load |
| Stomach | 0-100 |
| Duodenum | 100 |
| Jejunum | 100 |
| Proximal ileum | 1000 |
| Distal ileum | 10⁸ |
| Colon | 10¹² |

### Applicability of Enzyme Degradation for Industrial Scale THC-Glycoside Synthesis

The following example is provided to demonstrate the applicability of glycoside hydrolase digestion as an industrial processing step for the synthesis of selected THC-glycosides.

VB300X, which includes a relatively complex mixture of THC-glycosides obtained using biocatalytic glycosylation methods, was digested to provide a refined THC-glycoside mixture containing at least 95% VB311 and VB302 using Lallzyme Beta™ (Lallemand). Reactions containing 2 mg/ml THC-glycosides mixtures in 30% EtOH, 20 mM citrate buffer pH 4.0, and 5 mg/ml Lallzyme Beta™ were incubated at 44° C. with stirring. The reactions were monitored by HPLC and allowed to proceed until the desired refined THC-glycoside mixture was attained, at which time the reactions were stopped by changing the pH to 7.0 with 1M NaOH and decreasing the reaction temperature to minimize activity of the enzyme biocatalysts.

The resulting refined mixture of THC-glycosides is more amenable to downstream processing techniques for isolation and purification of the resulting glycosides. One such downstream processing technique that can be employed is solvent extraction using a cyclohexane-rich solvent to preferentially extract the VB302, but leaving behind the VB311 and higher THC-glycosides. Upon multiple cyclohexane-rich solvent extractions of the VB302/ VB311 mixture, the VB302 can be largely removed from the mixture. Following removal of VB302 from the mixture, the VB311 can then be solvent extracted using multiple rounds of ethyl acetate with ethanol to extract from the aqueous mixture. The purified VB302 or VB311 in the extraction solvents can then be evaporated and concentrated for further processing and purification.

Cyclohexane-rich solvent mixtures include varying ratios of cyclohexane to ethyl acetate. Higher glycosides are relatively insoluble in cyclohexane, so addition of cyclohexane to another solvent will decrease the extraction or uptake of higher glycosides like VB311. VB302 and other monosides are still relatively soluble in cyclohexane-rich solvent mixtures, so an aqueous solution containing only VB302 and VB311 can be differentially solvent extracted using an initial extraction with cyclohexane-rich solvent to remove the VB302, then followed with ethyl acetate or similar to extract the remaining VB311.

Multiple ratios of cyclohexane to ethyl acetate were tested for their effectiveness in separating different glycosidic mixtures, as reported in Table 15.

TABLE 15
Comparison of ethyl acetate:cyclohexane ratios

| | Ethyl Acetate:Cyclohexane Ratio | | |
|---|---|---|---|
| | 3:4 | 1:1 | 5.5:4.5 |
| VB311 Purity | 84.82% | 87.54% | 86.01% |
| VB311 Recovered | 115.01% | 106.06% | 98.05% |
| VB310 Removed | 31.86% | 58.56% | 66.18% |
| VB302 Removed | 99.09% | 99.81% | 99.81% |

It was found that 3:4 ethyl acetate:cyclohexane was superior for increased VB311 extraction yield while still maintaining high purity. Other solvent ratios were successful for preferential extraction of VB302. For example, to optimize VB311 purity over total yields, the ratio of ethyl acetate to cyclohexane can go to 1:1 or beyond. This is due to removal of VB310, which is the most significant impurity following digestion of VB300X mixed glycosides with Lallzyme Beta™ glycoside hydrolases.

These solvent extractions were carried out at lab-scale separatory funnel scale then transferred to pilot scale with a CINC VO2 centrifugal countercurrent liquid-liquid extractor.

This example demonstrates that a complicated mixture of THC-glycosides can be digested by a carbohydrate-digesting enzyme such as Lallzyme Beta™, to produce a relatively pure mixture of VB302 and VB311, which can be easily separated by differential solvent extraction, as previously described.

These novel sugar linkages on cannabinoid glycosides are beneficial due to extensive research on 1-4 and 1-6 linked carbohydrates in the biofuels and starch industries, and through the availability of commercial processing enzymes that assist in the preparation of specific cannabinoid glycoside structures for characterization and pharmaceutical use. By coupling enzymatic digestion of glycoside mixtures with simple solvent extraction of the resulting cannabinoid glycosides, a novel and valuable process for the efficient and cost effective production of selected cannabinoid glycosides has been developed.

The glycosidic linkages described herein are advantageous over previously described cannabinoid-glycosides for the aforementioned reasons.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

### REFERENCES

Bartzokis G. (2004). Age-related myelin breakdown: a developmental model of cognitive decline and Alzheimer's disease. Neurobiology of Aging. 25:5-18.

Bisogno T, et al. (2001) Molecular targets for cannabidiol and its synthetic analogues: effect on vanilloid VR₁ receptors and on the cellular uptake and enzymatic hydrolysis of anandamide. British Journal of Pharmacology. 134, 845-852.

Bowes, J. et al. “Reducing safety-related drug attrition: the use of in vitro pharmacological profiling.” Nature Reviews Drug Discovery 11, no. 12 (2012): 909

Chen Q, et al. (2009). Synthesis, in vitro and in vivo characterization of glycosyl derivatives of ibuprofen as novel prodrugs for brain drug delivery. J Drug Targeting. 17(4):318-328.

Conchie J., Findlay J., Levvy G A. (1958). Mammalian Glycosidases, Distribution in the body. Biochem J. 71(2):318-325.

De Petrocellis L, et al. (2011) Effects of cannabinoids and cannabinoid-enriched Cannabis extracts on TRP channels and endocannabinoid metabolic enzymes. British Journal of Pharmacology. 163, 1479-1494.

Dewitte G, et al. (2016) Screening of Recombinant Glycosyltransferases Reveals the Broad acceptor Specificity of Stevia UGT-76G1. Journal of Biotechnology. Accepted Manuscript, DOI: http://dx.doi.org/doi:10.1016/j.jbiotec.2016.06.034.

Friend D R., Chang G W. (1984). A Colon-Specific Drug-Delivery System Based on Drug Glycosides and the Glycosidases of the Colonic Bacteria. J Med Chem. 27:261-266.

Friend D R., Chang G W. (1985). Drug Glycosides: Potential Prodrugs for Colon-Specific Drug Delivery. J Med Chem. 28:51-57.

Gomez O., Arevalo-Martin A., Garcia-Ovejero D., Ortega-Gutierrez S., Cisneros J A., Almazan G, Sanchez-Rodriguez M A., Molina-Holgado F., Molina-Holgado E. (2010). The Constitutive Production of the Endocannabinoid 2-Arachidonoylglycerol Participates in Oligodendrocyte Differentiation. Glia. 58:1913-1927.

luvone T., Esposito G., De Filippis D., Scuderi C., Steardo L. (2009). Cannabidiol: a promising drug for neurodegenerative disorders? CNS Neurosci Ther. 15(1):65-75.

Jarh, P., Pate DW., Brenneisen R., Jarvinen T. (1998). Hydroxypropyl-beta-cyclodextrin and its combination with hydroxypropyl-methylcellulose increases aqueous solubility of delta9-tetrahydrocannabinol. Life Sci. 63(26):PL381-381.

Jiang R, et al. (2011) Identification of cytochrome P450 enzymes responsible for metabolism of cannabidiol by human liver microsomes. Life Sciences. 89, 165-170.

Kren V (2008) Glycoside vs. Aglycon: The Role of Glycosidic Residue in Biologic Activity. Glycoscience. pp2589-2644.

Kren V, Rezanka T (2008) Sweet antibiotics—the role of glycosidic residues in antibiotic and antitumor activity and their randomization. FEMS Microbiol Rev. 32, 858-889.

Li S., Li W., Xiao Q., Xia Y. (2012). Transglycosylation of stevioside to improve the edulcorant quality by lower substitution using cornstarch hydrolyzate and CGTase. J Food Chem. 138(2013):2064-2069.

Mazur A., et al. (2009). Characterization of Human Hepatic and Extrahepatic UDP-Glucuronosyltransferase Enzymes Involved in the Metabolism of Classic Cannabinoids. Drug Metabolism and Disposition. 37(7):1496-1504.

McPartland, John M., Christa MacDonald, Michelle Young, Phillip S. Grant, Daniel P. Furkert, and Michelle Glass. “Affinity and efficacy studies of tetrahydrocannabinolic acid A at cannabinoid receptor types one and two.” Cannabis and cannabinoid research 2, no. 1 (2017): 87-95.

Mecha M., Torrao AS., Mestre L., Carrillo-Salinas FJ., Mechoulam R., Guaza C. (2012). Cannabidiol protects oligodendrocyte progenitor cells from inflammation-induced apoptosis by attenuating endoplasmic reticulum stress. Cell Death and Disease. 3(e331).

Mechoulam R., Parker L A., Gallily R. (2002). Cannabidiol: An Overview of Some Pharmacological Aspects. 42(S1):11S-19S.

Mighdoll M I., Tao R., Kleinman J E., Hyde T M. (2015). Myelin, myelin-related disorders, and psychosis. Schizophr Res. 161(1):85-93.

Molina-Holgado E., Vela JM., Arevalo-Martin A., Almazan G., Molina-Holgado F., Borrell J., Guaza C. (2002). Cannabinoids Promote Oligodendrocyte Progenitor Survival: Involvement of Cannabinoid Receptors and Phosphatidylinositol-3-Kinase/Akt Signaling. J. Neurosci. 22(22):9742-9753.

Noguchi A, et al. (2009). Identification of an inducible glucosyltransferase from Phytolacca americana L. cells that are capable of glycosylating capsaicin. Plant Biotechnology. 26, 285-292.

Pacher P, et al. (2006) The endocannabinoid system as an emerging target of pharmacotherapy. Pharmacology Review. 58(3), 389-462.

Pertwee, Roger G. “Pharmacological actions of cannabinoids” in Cannabinoids, pp. 1-51. Springer, Berlin, Heidelberg, 2005.

Richman A., Swanso, A., Humphrey T., Chapman R., McGarvey B., Pocs R., Brandle J. (2005). Functional genomics uncovers three glucosyltransferases involved in the synthesis of the major sweet glucosides of Stevia rebaudiana. Plant J. 41(1):56-67.

Russo E., Guy, GW. (2006) A tale of two cannabinoids: the therapeutic rationale for combining tetrahydrocannabinol and cannabidiol. Medical Hypotheses. 66(2):234-46.

Sartor, B. (2008) Microbial Influences in Inflammatory Bowel Diseases. Gastroenterology, 134, no. 2 (2008): 577-594.

Tanaka H., et al. (1993). Cannabis, 21.¹ Biotransformation of cannabinol to its glycosides by in vitro plant tissue. Journal of Natural Products. 56(12):2068-2072.

Tanaka H., et al. (1996). Cannabis 25, biotransformation of cannabidiol and cannabidiolic acid by Pinellia ternata tissue segments. Plant Cell Reports. 15:819-823.

Terao J., Murota K., Kawai Y. (2011). Conjugated quercetin glucuronides as bioactive metabolites and precursors of aglycone in vivo. Food Function. 2:11-17.

Thomas A., et al. (2007) Cannabidiol displays unexpectedly high potency as an antagonist of CB, and CB₂ receptor agonists in vitro. British Journal of Pharmacology. 150, 613-623.

U.S. Pat. No. 8,410,064 B2. 2013. Classical cannabinoid metabolites and methods of use thereof.

U.S. Pat. No. 8,227,627 B2. 2012. Prodrugs of tetrahydrocannabinol, compositions comprising prodrugs of tetrahydrocannabinol and methods of using the same.

Watanabe K, et al. (1998) Distribution and characterization of anandamide amidohydrolase in mouse brain and liver. Life Sciences. 62(14), 1223-1229.

WO2009018389 A4. 2009. Prodrugs of cannabidiol, compositions comprising prodrugs of cannabidiol and methods of using the same.

WO2012011112 A1. 2011. Non psychoactive cannabinoids and uses thereof.

WO 2014108899 A1. 2014. Fluorinated CBD compounds, compositions and uses thereof.

Yamaori S, et al. (2011) Potent inhibition of human cytochrome P450 3A isoforms by cannabidiol: Role of phenolic hydroxyl groups in the resorcinol moiety. Life Sciences, 88, 730-736.

Zuardi A W, et al. (2012). A Critical Review of the Antipsychotic Effects of Cannabidiol: 30 Years of a Translational Investigation. Current Pharmaceutical Design, 18, 5131-5140.

1. A tetrahydrocannabinol glycoside prodrug compound having Formula (I): 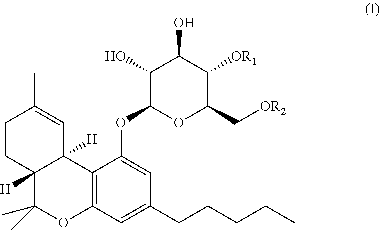
wherein
R₁ is H, β-D-glucopyranosyl, or 3-O-β-D-glucopyranosyl-β-D-glucopyranosyl; and
R₂ is H or β-D-glucopyranosyl;
with the proviso that R₁ and R₂ are not both H.

2. The compound of claim 1, having the structure:

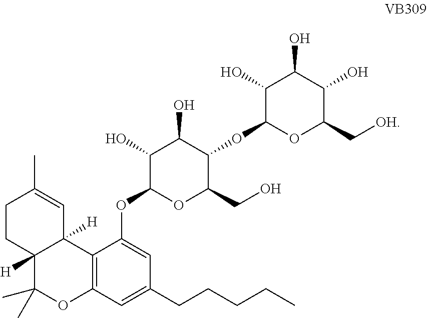 [SMILES: CCCCCc1cc(O[C@@H]2O[C@H](CO)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]

3. The compound of claim 1, having the structure:

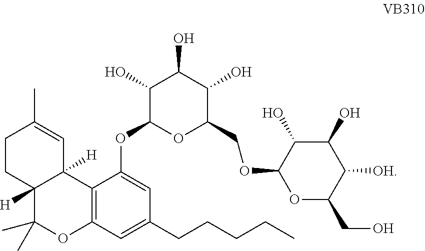 [SMILES: CCCCCc1cc(O[C@@H]2O[C@H](CO[C@@H]3O[C@H](CO)[C@@H](O)[C@H](O)[C@H]3O)[C@@H](O)[C@H](O)[C@H]2O)c2c(c1)OC(C)(C)[C@@H]1CCC(C)=C[C@@H]21]

4. The compound of claim 1, having the structure:

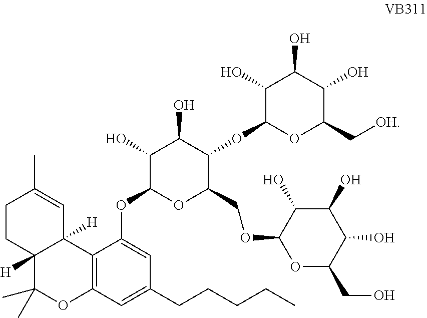 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]

5. The compound of claim 1, having the structure:

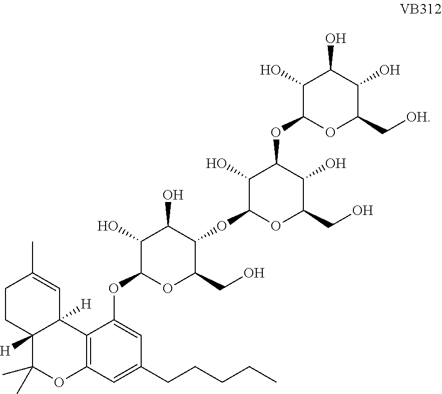 [SMILES: CCCCCc1cc(O[C@@H]2O[C@H](CO)[C@@H](O[C@@H]3O[C@H](CO)[C@@H](O)[C@H](O[C@@H]4O[C@H](CO)[C@@H](O)[C@H](O)[C@H]4O)[C@H]3O)[C@H](O)[C@H]2O)c2c(c1)OC(C)(C)[C@@H]1CCC(C)=C[C@@H]21]

6. The compound of claim 1, having the structure:

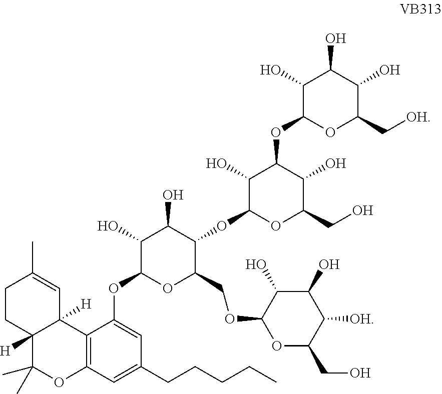 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O[C@@H]4O[C@H](CO)[C@@H](O)[C@H](O)[C@H]4O)[C@H]3O)[C@H](O)[C@H]2O)c2c(c1)OC(C)(C)[C@@H]1CCC(C)=C[C@@H]21]

7. A pharmaceutical composition comprising a compound as defined in claim 1 and a pharmaceutically acceptable carrier, diluent, excipient, adjuvant, or any combination thereof.

8. A method for the site-specific delivery of tetrahydrocannabinol to the intestinal lumen of a subject, comprising the step of administering a tetrahydrocannabinol glycoside prodrug as defined in claim 1 to a subject in need thereof.

9. (canceled)

10. (canceled)

11. A method for the site-specific delivery of tetrahydrocannabinol to the intestinal lumen of a subject, comprising the step of administering a pharmaceutical composition as defined in claim 7 to a subject in need thereof.

12. The method of claim 11, wherein the pharmaceutical composition is formulated for oral administration.

13. The method of claim 11, wherein the pharmaceutical composition is formulated for rectal administration.

14. A cannabidiol glycoside prodrug compound having Formula (II): 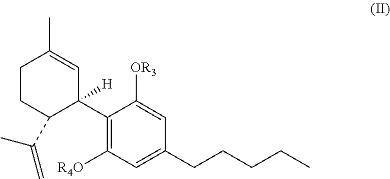
wherein R₃ and R₄ are H or a moiety having the structure: 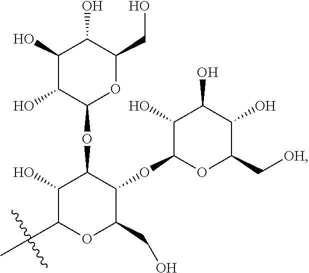 [SMILES: *C1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]
with the proviso that R₃ and R₄ are not both H.

15. The compound of claim 14, having the structure,

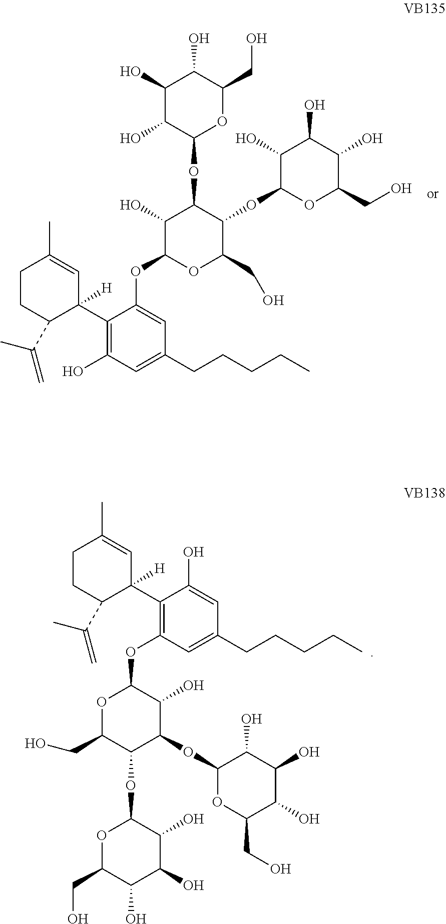 [SMILES: C=C(C)[C@@H]1CCC(C)=C[C@H]1c1c(O)cc(CCCCC)cc1O[C@@H]1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O ; C=C(C)[C@@H]1CCC(C)=C[C@H]1c1c(O)cc(CCCCC)cc1O[C@@H]1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]

16. A pharmaceutical composition comprising a compound as defined in claim 14 and a pharmaceutically acceptable carrier, diluent, excipient, adjuvant, or any combination thereof.

17. A method for the site-specific delivery of cannabidiol to the intestinal lumen of a subject, comprising the step of administering a cannabidiol glycoside prodrug as defined in claim 14 to a subject in need thereof.

18. (canceled)

19. (canceled)

20. A method for the site-specific delivery of a cannabinoid drug to the intestinal lumen of a subject, comprising the step of administering a pharmaceutical composition as defined in claim 16 to a subject in need thereof.

21. The method of claim 20, wherein the pharmaceutical composition is formulated for oral administration.

22. The method of claim 20, wherein the pharmaceutical composition is formulated for rectal administration.

23. A process for the preparation of a purified cannabinoid glycoside prodrug comprising the steps of:
(a) providing a mixture of higher order cannabinoid glycosides;
(b) incubating the mixture of cannabinoid glycosides with at least one hydrolase enzyme for a period of time sufficient to hydrolyze at least a portion of the glycosidic bonds to form a refined mixture of cannabinoid glycosides; and
(c) separating the purified cannabinoid glycoside prodrug from the refined mixture of cannabinoid glycosides.

24. The process of claim 23, wherein separation step further comprises the steps of:
extracting the refined mixture with extraction solvent to provide a solution of extracted cannabinoid glycoside prodrug, and
evaporating the extraction solvent to provide the purified cannabinoid glycoside prodrug.

25. The process of claim 24, wherein the extraction solvent is a mixture of ethyl acetate and cyclohexane.

26. The process of claim 23, wherein the cannabinoid glycosides are tetrahydrocannabinol glycosides.

27. The process of claim 26, wherein the mixture of higher order tetrahydrocannabinol glycosides comprises a mixture of

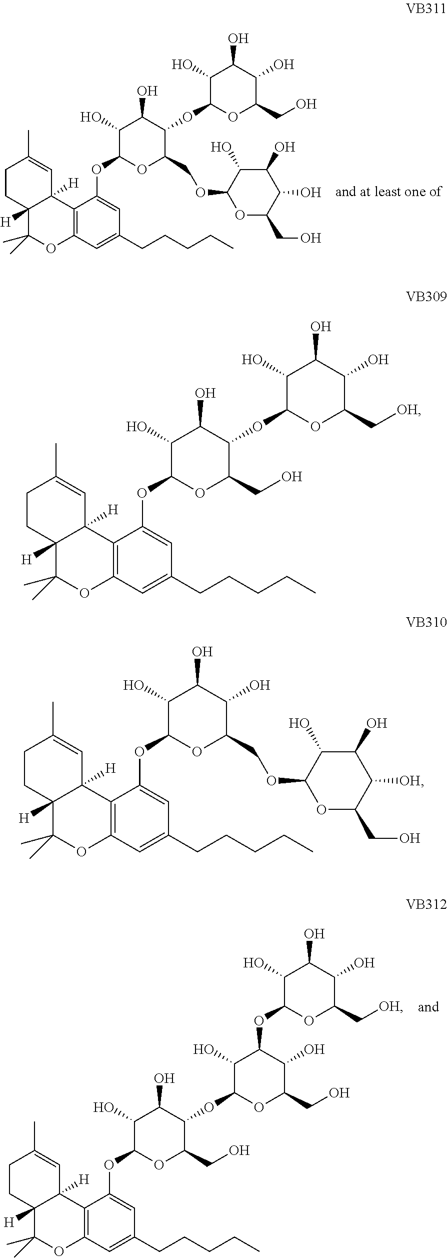

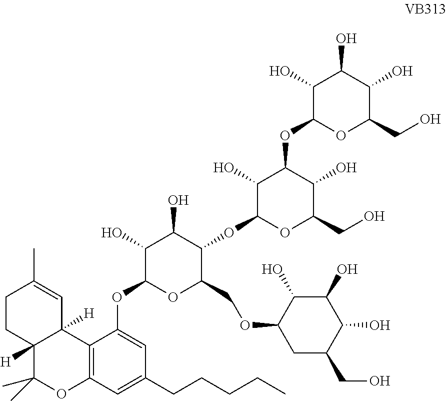 [SMILES: CCCCCc1cc(O[C@@H]2O[C@H](CO[C@@H]3C[C@H](CO)[C@@H](O)[C@H](O)[C@H]3O)[C@@H](O[C@@H]3O[C@H](CO)[C@@H](O)[C@H](O[C@@H]4O[C@H](CO)[C@@H](O)[C@H](O)[C@H]4O)[C@H]3O)[C@H](O)[C@H]2O)c2c(c1)OC(C)(C)[C@@H]1CCC(C)=C[C@@H]21]

28. The process of claim 27, wherein at least one hydrolase enzyme is Lallzyme Beta™.

29. The process of claim 27, wherein the purified tetrahydrocannabinol glycoside prodrug is

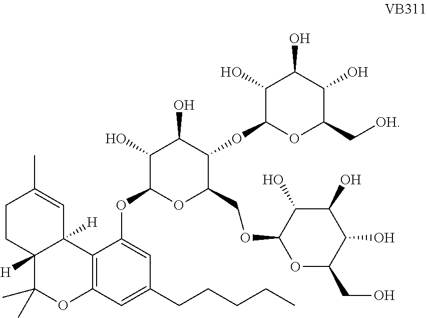 [SMILES: CCCCCc1cc(O[C@@H]2O[C@H](CO[C@@H]3O[C@H](CO)[C@@H](O)[C@H](O)[C@H]3O)[C@@H](O[C@@H]3O[C@H](CO)[C@@H](O)[C@H](O)[C@H]3O)[C@H](O)[C@H]2O)c2c(c1)OC(C)(C)[C@@H]1CCC(C)=C[C@@H]21]

30. The process of claim 23, wherein the cannabinoid glycosides are cannabidiol glycosides.

31. The process of claim 30, wherein the mixture of higher order cannabidiol glycosides comprises at least

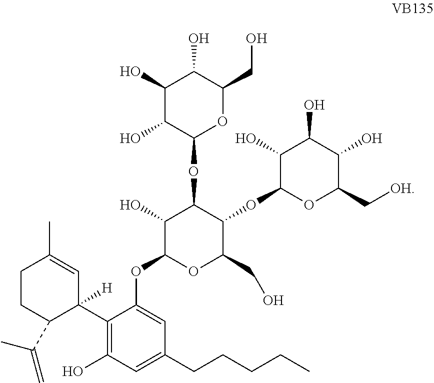 [SMILES: C=C(C)[C@@H]1CCC(C)=C[C@H]1c1c(O)cc(CCCCC)cc1O[C@@H]1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]

32. The process of claim 31, wherein at least one hydrolase enzyme is Vinotaste Pro®.

33. The process of claim 31, wherein the purified cannabidiol glycoside prodrug is 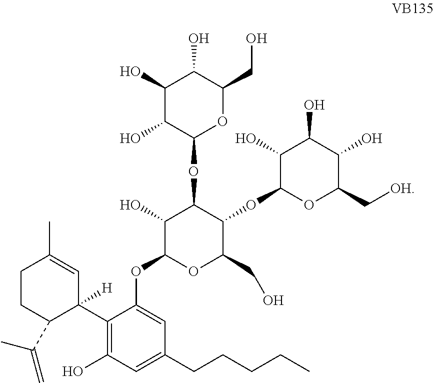 [SMILES: C=C(C)[C@@H]1CCC(C)=C[C@H]1c1c(O)cc(CCCCC)cc1O[C@@H]1O[C@H](CO)[C@@H](O[C@@H]2O[C@H](CO)[C@@H](O)[C@H](O)[C@H]2O)[C@H](O[C@@H]2O[C@H](CO)[C@@H](O)[C@H](O)[C@H]2O)[C@H]1O]
